(19) United States
(12) Patent Application Publication
(10) Pub. No.: US 2021/0275596 A1
(43) Pub. Date: Sep. 9, 2021

(54) APPLICATION OF GENE PROFILE FOR CELLS ISOLATED USING FRESH-TRACER

(71) Applicant: CELL2IN, INC., Seoul (KR)

(72) Inventors: Heun Soo KANG, Seoul (KR); Hye Mi KIM, Seoul (KR); Ji Eun SONG, Seoul (KR); Yingfu YIN, Seoul (KR); Ji Woong SHIN, Seoul (KR); Hye Won KANG, Seoul (KR); Yong Hwan KIM, Anyang-si Gyeonggi-do (KR)

(21) Appl. No.: 17/253,862

(22) PCT Filed: Jul. 20, 2018
(86) PCT No.: PCT/KR2018/008239

§ 371 Date: Dec. 18, 2020

(51) Int. Cl. A61K 35/28

(52) U.S. Cl. CPC A61K 35/28; A61K 2035/124

(57) ABSTRACT

The present invention relates to a therapeutic stem cell composition comprising a stem cell in which the expression level of any one or more genes selected from the group consisting of ACAN, SOX9, AP2, RUNX2, OCN, ALP, OCT4, SOX2, CXCR4, cMET, PDGFRA, PDGFRB, VEGF-R1, VEGF-R2, CSF-1, IDO2, Sox2, Nanog, cMyc, Klf2, Klf4, Rex1, Esrrb, Neurog1, Neurod1, Nkx2.2, Ascl2, Gfap, S100b, Olig2, Neurog2, T, Nkx2.5, Esrrbb, Klf2, Klf4, cTnT, a-Actin, Mlc2v and Runx1 is higher or lower than the expression level of GAPDH (glyceraldehyde 3-phosphate dehydrogenase) gene.

### CROSS-REFERENCE TO RELATED APPLICATIONS

This application is a U.S. National Stage Application of International Patent Application Number PCT/KR2018/008239, filed Jul. 20, 2018, which is hereby incorporated herein by reference in its entirety.

### TECHNICAL FIELD

The present invention relates to the application of gene profiles of cells sorted based on a FreSH-tracer.

### BACKGROUND ART

Reactive oxygen species (ROS) are important signaling molecules that regulate cellular metabolism, proliferation, and survival (Winterbourn and Hampton, 2008). An increase of ROS induces the thiol oxidation of cysteine residues on signaling proteins, resulting in alterations of protein activities to regulate cellular functions. In particular, ROS-mediated oxidation plays an important role in regulating a variety of signaling proteins in stem cells (SCs) that influence self-renewal capacity, pluripotency, viability, and genomic stability. These signaling proteins include OCT4, NRF2, FoxOs, APE1/Ref-1, ATM, HIVE-1, p38, and p53 (Wang et al., 2013). For example, disruption of Nrf2, a master regulator of redox homeostasis, impinges upon the functions of embryonic and adult SCs such as the self-renewal and pluripotency in ESCs (Jang et al., 2014), the migration and retention of hematopoietic SCs in the bone marrow niche (Tsai et al., 2013), and the proliferation and homeostasis in intestines (Hochmuth et al., 2011) and airway basal stem cells (Paul et al., 2014). Thus, the cellular redox regulation is critical for maintaining stemness and functional potency of embryonic stem cells and adult stem cells.

PCT International Patent Publication No. WO2013059829 A1 describes an approach that creates non-tumorigenic PSCs by treating human fibroblasts with the extracellular matrix component fibromodulin, which are characterized by expression of the core pluripotency factors nanog, oct4 and sox2 as well as the negative cell cycle regulators p15 and p21. Korean Patent Application Publication No. 10-2016-0062157 describes a method that reprograms pluripotent stem cells (PSCs) by epigenetic conditioning and metabolic reprogramming into pPSCs with highly controllable biological functions. However, the two approaches all relate to the inhibition of abnormal proliferation or tumorigenicity and differ from the present invention. In addition, the present invention differs in that it has identified specific gene expression ratios in stem cells such that the quality of the stem cells can be managed according to the characteristics thereof

Through the specification, a number of publications and patent documents are referred to and cited. The disclosure of the cited publications and patent documents is incorporated herein by reference in its entirety to more clearly describe the state of the technical field to which the present invention pertains and the present disclosure.

### DISCLOSURE

### Technical Problem

It is an object of the present invention to provide a therapeutic stem cell composition based on gene profiles of cells sorted by a FreSH-tracer.

Specifically, the present invention is intended to provide a therapeutic stem cell composition comprising stem cells, which are obtained by sorting according to their characteristics based on an identified specific gene expression ratio such that the necessary characteristics of the stem cells can be selectively managed.

Another object of the present invention is to provide a pharmaceutical composition, which comprises the above-described therapeutic stem cell composition and is to be administered to a subject suffering from asthma to alleviate, prevent or treat asthma.

Still another object of the present invention is to provide a pharmaceutical composition which comprises the above-described therapeutic stem cell composition and is to be administered to a subject suffering from allergic asthma to alleviate, prevent or treat allergic asthma

However, objects which are to be achieved by the present invention are not limited to the above-mentioned objects, and other objects of the present invention will be clearly understood by those skilled in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

As used herein, the term “FreSH-tracer (Fluorescent Real-time SH group-tracer)” means a compound represented by formula 1 below, which is a coumarin derivative having a cyanoacrylamide electrophile and is used as a fluorescent substance for measurement of cellular activity in the present invention. First, the FreSH-tracer of the present invention is brought into contact with cells. This is a step of labeling cells with the FreSH-tracer. In this step, the fluorescence intensity at 430-550 nm or 550-680 nm in the cells labeled with the FreSH-tracer, or the ratio of the fluorescence intensity at 430-550 nm to the fluorescence intensity at 550-680 nm in the cells, is measured in real time. According to one embodiment of the present invention, the fluorescence intensity at 430-550 nm is the fluorescence intensity at 450-550, 470-550, 470-530, 490-530, 500-520 or 510 nm. According to one embodiment of the present invention, the fluorescence intensity at 550-680 nm is the florescence intensity at 550-650, 550-620, 550-600, 570-590 or 580 nm. As demonstrated in examples below, cells of the present invention, in which the ratio of the fluorescence intensity at 430-550 nm to the fluorescence intensity at 430-550 nm or 550-680 nm is high or the fluorescence intensity at 550-680 nm is low, have high glutathione (GSH) activity and high cellular antioxidant activity, indicating that cellular antioxidant activity can be measured by the method of the present invention

A method for measuring the antioxidant activity (or anti-oxidative activity) of stem cells comprises the steps of (a) brining a composition for measuring cell activity, which comprises a compound represented by the following formula 1 or a salt thereof, into contact with cells; and (b) observing in real-time (i) the fluorescence intensity at 430-550 nm or 550-680 nm in the cells, or (ii) the ratio of the fluorescence intensity at 430-550 nm to the fluorescence intensity at 550-680 nm in the cells:

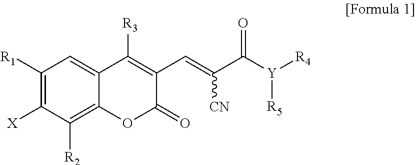

wherein R₁ and R₂ are each independently hydrogen or C₁₋₄straight-chain or branched alkyl, or R₁ and R₂together with X form a five- or six- membered heterocycloalkyl or heterocycloalkenyl ring; R₃ is hydrogen or C₁₄ straight-chain or branched alkyl; R₄ and R₅ are each independently hydrogen, C₁₋₅ straight-chain or branched alkyl, or —(CH₂)ₘ—COO—C₁₋₅straight-chain or branched alkyl (where m is an integer ranging from 1 to 5), or R₄ and R₅ together with Y form a C₃₋₇heterocycloalkyl which may be unsubstituted or substituted with R₆; R₆ is —COO(CH₂)ₙ—OCO—C₁₋₅straight-chain or branched alkyl (where n is an integer ranging from 1 to 5), —(CONH)—(CH₂)ₒ—PPh₃⁺Cl⁻ (where o is an integer ranging from 1 to 5), or —(CONH)—CHR₇—COO(CH₂)ₚ—OCO—C₁₋₅straight-chain or branched alkyl (where p is an integer ranging from 1 to 5); R₇ is —(CH₂)_{q}—COO(CH₂)—OCO—C₁₋₅straight-chain or branched alkyl (where q and r are each an integer ranging from 1 to 5); and X and Y are each independently N or O.

According to one embodiment of the present invention, the compound represented by formula 1 is preferably a compound selected from the group consisting of compounds represented by formulas 2 to 7 below. According to another embodiment of the present invention, the compound represented by formula 1 is more preferably a compound represented by the following formula 2:

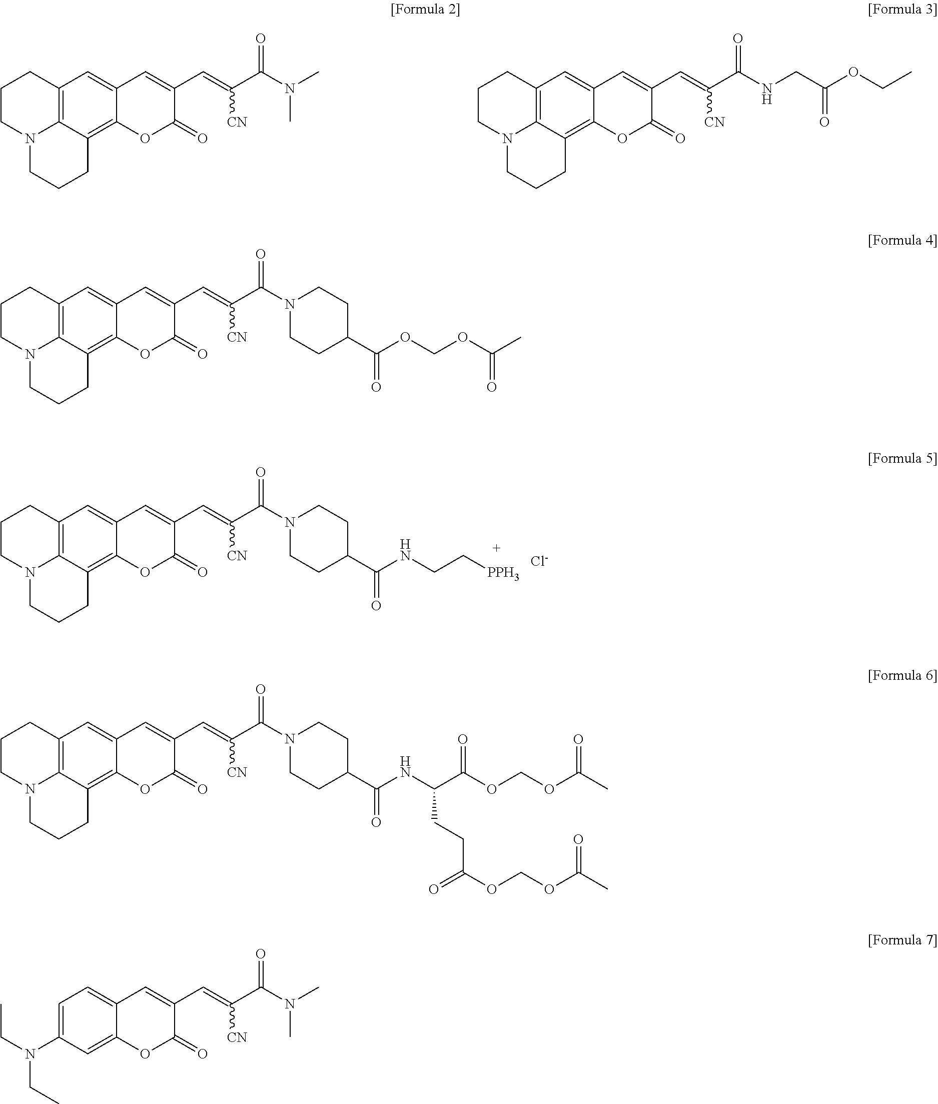

As used herein, the term “antioxidant activity” means the ability to restore antioxidant activity, which is expression of the intrinsic function of cells.

Reactive oxygen species (ROS) are chemically reactive molecules containing oxygen. The term “ROS signaling” refers to the process of ROS generated during aerobic metabolism typically by oxidative phosphorylation, which act as second messengers in cellular signaling. ROS are essential regulators of cellular metabolism and are generated in virtually all cells either by the mitochondrial electron transport chain or by NADPH oxidase. Oxidative phosphorylation is required for aerobic metabolism. During the process of oxidative phosphorylation, the oxidoreduction energy generated over the mitochondrial electron transport chain is bound in a high energy phosphate group in the form of ATP. Cytochrome c oxidase is the final component in the electron transport chain and catalyzes the reduction of oxygen (O₂) to water (H₂O), where oxygen serves as the final electron acceptor. However incomplete reduction of oxygen does also occur and leads to the generation of highly reactive oxygen metabolites which include superoxide radicals (O₂) and hydrogen peroxide (H₂O₂), while hydroxyl radicals (OH*) can form in the presence of transition metal ions. These partially reduced oxygen species are described as ROS. If unchecked by antioxidative enzyme systems of the cells, ROS can have deleterious effects and lead to cellular damage, aging and cell death. However, ROS are also involved in non-deleterious cellular processes and play an important regulatory role in the cell. For example, oxidation of transcription factors by hydrogen peroxide can lead to a conformational change and direct activation of gene expression. The current paradox of ROS signaling is that too much ROS damage the cell by oxidation of vital cellular components, but a lack of or too little ROS impairs important physiological functions and cellular signaling mechanisms. Therefore, ROS signaling is a highly regulated and balanced system in the cell.

In the present invention, glyceraldehyde 3-phosphate dehydrogenase is also abbreviated as GAPDH or G3PDH.

As used herein, the term “stem cell” means an undifferentiated cell having self-renewal ability and differentiation/proliferation ability. Stem cells include subpopulations, such as pluripotent stem cells, multipotent stem cells, unipotent stem cells and the like, according to the differentiation ability. The pluripotent stem cell means a cell capable of differentiating into any tissue or cell constituting living organisms. Furthermore, the multipotent stem cell means a cell capable of differentiating into plural, though not all, kinds of tissues and cells. The unipotent stem cell means a cell capable of differentiating into specific tissues and cells. Examples of the pluripotent stem cell include embryonic stem cells (ES cells), embryonic germ cell (EG cells), induced pluripotent stem cell (iPS cells), and the like. Examples of the multipotent stem cell include adult stem cells, such as mesenchymal stem cells (derived from adipose, bone marrow, umbilical cord blood, umbilical cord, or the like), haematopoietic stem cells (derived from bone marrow, peripheral blood, or the like), neural stem cells, germ stem cells, and the like. In addition, examples of the unipotent stem cell include committed stem cells which are normally quiescent with low self-renewal capacity, but vigorously differentiate into hepatocytes under certain conditions. Particularly, in the present invention, mesenchymal stem cells (MSCs) are preferably hES-MSCs (human embryonic stem cell-derived mesenchymal stroma cells), BM-MSCs (bone marrow mesenchymal stem cells), UC-MSCs (umbilical cord mesenchymal stem cells), and ADSC (adipose derived stem cell-conditioned medium), but are not limited thereto.

As used herein, the term “embryonic stem cells (ESCs)” refers to cells obtained by isolating inner cell mass from blastocysts immediately before embryo implantation and culturing the isolated inner cell mass in vitro. The stem cells have pluripotency to differentiate into cells of all tissues of the body. In a broad sense, the term “stem cell” includes an embryonic body or embryoid body derived from embryonic stem cells. As used herein, the term “embryonic body or embryoid body (EB)” refers to a spherical stem cell mass generated in a suspension culture condition, and has the potential to differentiate into endoderm, mesoderm and ectoderm, and thus is used as a precursor in most differentiation inducing processes to obtain tissue-specific differentiated cells.

To achieve the above-described objects, the present invention provides a therapeutic stem cell in which the expression level of any one or more selected from the group consisting of ACAN, SOX9, AP2, RUNX2, OCN, ALP, OCT4, SOX2, CXCR4, cMET, PDGFRA, PDGFRB, VEGF-R1, VEGF-R2, CSF-1, IDO2, Sox2, Nanog, cMyc, Klf2, Klf4, Rex1, Esrrb, Neurog1,Neurod1, Nkx2.2, Ascl2, Gfap, S100b, Olig2, Neurog2, T, Nkx2.5, Esrrbb, Klf2, Klf4, cTnT, a-Actin, Mlc2v, and Runx1, is higher or lower than the expression level of GAPDH (glyceraldehyde 3-phosphate dehydrogenase) gene.

In one embodiment of the present invention, the stem cell is any one selected from the group consisting of mesenchymal stem cell (MSC), embryonic stem cell (ESC), or embryoid body (EB). In another embodiment of the present invention, the stem cell is a mesenchymal stem cell (MSC) in which the expression level of any one or more genes selected from the group consisting of AP2 and ALP is higher than the expression level of the GAPDH (glyceraldehyde 3-phosphate dehydrogenase) gene. In still another embodiment of the present invention, the stem cell is cultured in any one medium selected from the group consisting of adipogenic induction medium, osteogenic induction medium, or chondrogenic induction medium. In still another embodiment of the present invention, the stem cell is a mesenchymal stem cell (MSC) in which the expression level of any one or more genes selected from the group consisting of ACAN, SOX9, RUNX2 and OCN is lower than the expression level of the GAPDH (glyceraldehyde 3-phosphate dehydrogenase) gene. In still another embodiment of the present invention, the stem cell is cultured in any one medium selected from the group consisting of adipogenic induction medium, osteogenic induction medium, or chondrogenic induction medium. In still another embodiment of the present invention, the stem cell is a mesenchymal stem cell (MSC) in which the expression level of any one or more genes selected from the group consisting of OCT4, SOX2, CXCR4, cMET, PDGFRA, PDGFRB, VEGF-R1, VEGF-R2, CSF-1 and IDO2 is lower than the expression level of the GAPDH (glyceraldehyde 3-phosphate dehydrogenase) gene. In still another embodiment of the present invention, the stem cell is an embryonic stem cell (ESC) in which the expression level of any one or more genes selected from the group consisting of Oct4, Sox2, Nanog, Klf2, Rex1 and Esrrb is higher than the expression level of the GAPDH (glyceraldehyde 3-phosphate dehydrogenase) gene. In still another embodiment of the present invention, the stem cell is an embryonic stem cell (ESC) in which the expression level of any one or more genes selected from the group consisting of cMyc, Klf4, Neurog1, Neurod1, Nkx2.2, Ascl2, Gfap, S100b and Olig2 is lower than the expression level of the GAPDH (glyceraldehyde 3-phosphate dehydrogenase) gene. In still another embodiment of the present invention, the stem cell is an embryoid body (EB) in which the expression level of any one or more genes selected from the group consisting of Oct4, Esrrbb and Klf2 is higher than the expression level of the GAPDH (glyceraldehyde 3-phosphate dehydrogenase) gene in the initial stage of culture of the embryoid body. In still another embodiment of the present invention, the stem cell is an embryoid body (EB) in which the expression level of any one or more genes selected from the group consisting of Neurog2, Olig2, T, Nkx2.5, Klf4, cTnT, a-Actin, Mlc2v and Runx1 is lower than the expression level of the GAPDH (glyceraldehyde 3-phosphate dehydrogenase) gene in the initial stage of culture of the embryoid body. In still another embodiment of the present invention, the stem cell is an embryoid body (EB) in which the expression level of any one or more genes selected from the group consisting of T and Oct4 is higher than the expression level of the GAPDH (glyceraldehyde 3-phosphate dehydrogenase) gene on 1 to 6 days of culture of the embryoid body. In still another embodiment of the present invention, the stem cell is an embryoid body (EB) in which the expression level of any one or more genes selected from the group consisting of Neurog2, Olig2, Nkx2.5, Esrrbb, Klf2, Klf4, cTnT, a-Actin, Mlc2v and Runx1 is lower than the expression level of the GAPDH (glyceraldehyde 3-phosphate dehydrogenase) gene on 1 to 6 days of culture of the embryoid body. In still another embodiment of the present invention, the stem cell is an embryoid body (EB) in which the expression level of any one or more genes selected from the group consisting of cTnT, a-Actin and Mlc2v is higher than the expression level of the GAPDH (glyceraldehyde 3-phosphate dehydrogenase) gene on 7 to 8 days of culture of the embryoid body. In still another embodiment of the present invention, the stem cell is an embryoid body (EB) in which the expression level of any one or more genes selected from the group consisting of Neurog2, Olig2, T, Nkx2.5, Oct4, Esrrbb, Klf2, Klf4 and Runx1 is lower than the expression level of the GAPDH (glyceraldehyde 3-phosphate dehydrogenase) gene on 7 to 8 days of culture of the embryoid body. In still another embodiment of the present invention, the stem cell is a mesenchymal stem cell (MSC) in which the expression level of ACAN is 0.0009- to 0.0012-fold of that of GAPDH after chondrogenic differentiation. In still another embodiment of the present invention, the stem cell is cultured in any one medium selected from the group consisting of adipogenic induction medium, osteogenic induction medium, or chondrogenic induction medium. In still another embodiment of the present invention, the stem cell is a mesenchymal stem cell (MSC) in which the expression level of SOX9 is 0.006- to 0.0077-fold of that of GAPDH after chondrogenic differentiation. In still another embodiment of the present invention, the stem cell is cultured in any one medium selected from the group consisting of adipogenic induction medium, osteogenic induction medium, or chondrogenic induction medium.

In still another embodiment of the present invention, the stem cell is a mesenchymal stem cell (MSC) in which the expression level of AP2 is 9.3- to 11.4-fold of that of GAPDH after adipogenic differentiation. In still another embodiment of the present invention, the stem cell is cultured in any one medium selected from the group consisting of adipogenic induction medium, osteogenic induction medium, or chondrogenic induction medium. In still another embodiment of the present invention, the stem cell is a mesenchymal stem cell (MSC) in which the expression level of RUNX2 is 0.38- to 0.48-fold of that of GAPDH after osteogenic differentiation. In still another embodiment of the present invention, the stem cell is cultured in any one medium selected from the group consisting of adipogenic induction medium, osteogenic induction medium, or chondrogenic induction medium. In still another embodiment of the present invention, the stem cell is a mesenchymal stem cell (MSC) in which the expression level of OCN is 0.074- to 0.092-fold of that of GAPDH after osteogenic differentiation. In still another embodiment of the present invention, the stem cell is cultured in any one medium selected from the group consisting of adipogenic induction medium, osteogenic induction medium, or chondrogenic induction medium. In still another embodiment of the present invention, the stem cell is the mesenchymal stem cell (MSC) in which the expression level of ALP is 27.6- to 33.8-fold of that of GAPDH after osteogenic differentiation. In still another embodiment of the present invention, the stem cell is cultured in any one medium selected from the group consisting of adipogenic induction medium, osteogenic induction medium, or chondrogenic induction medium. In still another embodiment of the present invention, the stem cell is a mesenchymal stem cell (MSC) whose multipotency increases when the expression level of OCT4 is 0.0051- to 0.0063-fold of that of GAPDH. In still another embodiment of the present invention, the stem cell is a mesenchymal stem cell (MSC) whose multipotency increases when the expression level of SOX2 is 0.0099- to 0.0122-fold of that of GAPDH. In still another embodiment of the present invention, the stem cell is a mesenchymal stem cell (MSC) whose migration is promoted when the expression level of CXCR4 is 0.0062- to 0.0077-fold of that of GAPDH. In still another embodiment of the present invention, the stem cell is a mesenchymal stem cell (MSC) whose growth and proliferation are promoted when the expression level of MET is 0.063- to 0.078-fold of that GAPDH. In still another embodiment of the present invention, the stem cell is a mesenchymal stem cell (MSC) whose engraftment rate, viability and vascular regeneration ability increase when the expression level of PDGFRA is 0.31- to 0.39-fold of that of GAPDH. In still another embodiment of the present invention, the stem cell is a mesenchymal stem cell (MSC) whose engraftment rate, viability and vascular regeneration ability increase when the expression level of PDGFRB is 0.45- to 0.56-fold of that of GAPDH. In still another embodiment of the present invention, the stem cell is a mesenchymal stem cell (MSC) whose engraftment rate, viability and vascular regeneration ability increase when the expression level of VEGF-R1 is 0.44- to 0.55-fold of that of GAPDH. In still another embodiment of the present invention, the stem cell is a mesenchymal stem cell (MSC) whose engraftment rate, viability and vascular regeneration ability increase when the expression level of VEGF-R2 is 0.62- to 0.77-fold of that of GAPDH. In still another embodiment of the present invention, the stem cell is a mesenchymal stem cell (MSC) whose immunomodulatory effect is enhanced when the expression level of CSF-1 is 0.19- to 0.24-fold of that of GAPDH. In still another embodiment of the present invention, the stem cell is a mesenchymal stem cell (MSC) whose anti-inflammatory effect is enhanced when the expression level of IDO2 is 0.00131- to 0.00160-fold of that of of GAPDH. In still another embodiment of the present invention, the stem cell is an embryonic stem cell (ESC) whose pluripotency increases when the expression level of Oct4 is 21.4- to 26.2-fold of that of GAPDH. In still another embodiment of the present invention, the stem cell is an embryonic stem cell (ESC) whose pluripotency increases when the expression level of Sox2 is 3.3- to 4.1-fold of that of GAPDH. In still another embodiment of the present invention, the stem cell is an embryonic stem cell (ESC) whose pluripotency increases when the expression level of Nanog is 3.7- to 4.6-fold of that of GAPDH. In still another embodiment of the present invention, the stem cell is an embryonic stem cell (ESC) whose pluripotency increases when the expression level of cMyc is 0.8- to 1.1-fold of that of GAPDH. In still another embodiment of the present invention, the stem cell is an embryonic stem cell (ESC) whose pluripotency increases when the expression level of Klf2 is 10.6- to 13.1-fold of that of GAPDH. In still another embodiment of the present invention, the stem cell is an embryonic stem cell (ESC) whose pluripotency increases when the expression level of Klf4 is 0.64- to 0.79-fold of that of GAPDH. In still another embodiment of the present invention, the stem cell is an embryonic stem cell (ESC) whose pluripotency increases when the expression level of Rex1 is 8.5- to 10.4-fold of that of GAPDH. In still another embodiment of the present invention, the stem cell is an embryonic stem cell (ESC) whose pluripotency increases when the expression level of Esrrb is 2.4- to 3.0-fold of that of GAPDH. In still another embodiment of the present invention, the stem cell is an embryonic stem cell (ESC) whose neuronal differentiation is promoted when the expression level of Neurogl is 0.49- to 0.60-fold of that of GAPDH. In still another embodiment of the present invention, the stem cell is an embryonic stem cell (ESC) whose neuronal differentiation is promoted when the expression level of Neurodl is 0.17- to 0.22-fold of that of GAPDH. In still another embodiment of the present invention, the stem cell is an embryonic stem cell (ESC) whose neuronal differentiation is promoted when the expression level of Nkx2.2 is 0.00064- to 0.00080-fold of that of GAPDH. In still another embodiment of the present invention, the stem cell is an embryonic stem cell (ESC) whose neuronal differentiation is promoted when the expression level of Ascl2 is 0.16-fold to 0.21-fold of that of GAPDH. In still another embodiment of the present invention, the stem cell is the embryonic stem cell (ESC) whose neuronal differentiation is promoted when the expression level of Gfap is 0.13- to 0.17-fold of that of GAPDH. In still another embodiment of the present invention, the stem cell is an embryonic stem cell (ESC) whose neuronal differentiation is promoted when the expression level of S100b is 0.012- to 0.016-fold of that of GAPDH. In still another embodiment of the present invention, the stem cell is an embryonic stem cell (ESC) whose neuronal differentiation is promoted when the expression level of Olig2 is 0.025- to 0.032-fold of that of GAPDH. In still another embodiment of the present invention, the stem cell is a stem cell forming an embryoid body (EB) whose pluripotency and differentiation ability increase when the expression level of Neurog2 is 0.00008- to 0.00010-fold of that of GAPDH in the initial stage of culture of the embryoid body. In still another embodiment of the present invention, the stem cell is a stem cell forming an embryoid body (EB) whose pluripotency and differentiation ability increase when the expression level of Olig2 is 0.0047- to 0.0059-fold of that of GAPDH in the initial stage of culture of the embryoid body. In still another embodiment of the present invention, the stem cell is a stem cell forming an embryoid body (EB) whose pluripotency and differentiation ability increase when the expression level of T is 0.036- to 0.045-fold of that of GAPDH in the initial stage of culture of the embryoid body. In still another embodiment of the present invention, the stem cell is a stem cell forming an embryoid body (EB) whose pluripotency and differentiation ability increase when the expression level of Nkx2.5 is 0.0043- to 0.0053-fold of that of GAPDH in the initial stage of culture of the embryoid body. In still another embodiment of the present invention, the stem cell is a stem cell forming an embryoid body (EB) whose pluripotency and differentiation ability increase when the expression level of Oct4 is 17.84- to 21.81-fold of that of GAPDH in the initial stage of culture of the embryoid body. In still another embodiment of the present invention, the stem cell is a stem cell forming an embryoid body (EB) whose pluripotency and differentiation ability increase when the expression level of Esrrbb is 2.74- to 3.36-fold of that of GAPDH in the initial stage of culture of the embryoid body. In still another embodiment of the present invention, the stem cell is a stem cell forming an embryoid body (EB) whose pluripotency and differentiation ability increase when the expression level of Klf2 is 10.10- to 12.50-fold of that of GAPDH in the initial stage of culture of the embryoid body. In still another embodiment of the present invention, the stem cell is a stem cell forming an embryoid body (EB) whose pluripotency and differentiation ability increase when the expression level of Klf4 is 0.60- to 0.73-fold of that of

GAPDH in the initial stage of culture of the embryoid body. In still another embodiment of the present invention, the stem cell is a stem cell forming an embryoid body (EB) whose pluripotency and differentiation ability increase when the expression level of cTnT is 0.0009- to 0.0011-fold of that of GAPDH in the initial stage of culture of the embryoid body. In still another embodiment of the present invention, the stem cell is a stem cell forming an embryoid body (EB) whose pluripotency and differentiation ability increase when the expression level of a-Actin is 0.36- to 0.45-fold of that of GAPDH in the initial stage of culture of the embryoid body. In still another embodiment of the present invention, the stem cell is a stem cell forming an embryoid body (EB) whose pluripotency and differentiation ability increase when the expression level of Mlc2v is 0.050- to 0.062-fold of that of GAPDH in the initial stage of culture of the embryoid body. In still another embodiment of the present invention, the stem cell is a stem cell forming an embryoid body (EB) whose pluripotency and differentiation ability increase when the expression level of Runxl is 0.016- to 0.021-fold of that of GAPDH in the initial stage of culture of the embryoid body. In still another embodiment of the present invention, the stem cell is a stem cell forming an embryoid body (EB) whose pluripotency and differentiation ability increase when the expression level of Neurog2 is 0.00030- to 0.00042-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Olig2 is 0.00050- to 0.00072-fold of that of GAPDH on 1 to 6 days of culture of the embryoid body. In still another embodiment of the present invention, the stem cell is a stem cell forming an embryoid body (EB) whose pluripotency and differentiation ability increase when the expression level of T is 2.60- to 3.19-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Nkx2.5 is 0.0070- to 0.0090-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Oct4 is 1.20- to 1.60-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Esrrbb is 0.008- to 0.011-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Klf2 is 0.16- to 0.20-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Klf4 is 0.067- to 0.083-fold of that of GAPDH on 1 to 6 days of culture of the embryoid body. In still another embodiment of the present invention, the stem cell is a stem cell forming an embryoid body (EB) whose pluripotency and differentiation ability increase when the expression level of cTnT is 0.020- to 0.025-fold of that of GAPDH on 1 to 6 days of culture of the embryoid body. In still another embodiment of the present invention, the stem cell is a stem cell forming an embryoid body (EB) whose pluripotency and differentiation ability increase when the expression level of a-Actin is 0.15- to 0.20-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Mlc2v is 0.11- to 0.14-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Runxl is 0.089- to 0.110-fold of that of GAPDH on 1 to 6 days of culture of the embryoid body. In still another embodiment of the present invention, the stem cell is a stem cell forming an embryoid body (EB) whose pluripotency and differentiation ability increase when the expression level of Neurog2 is 0.00078- to 0.0010-fold of that of GAPDH on 7 to 8 days of culture of the embryoid body. In still another embodiment of the present invention, the stem cell is a stem cell forming an embryoid body (EB) whose pluripotency and differentiation ability increase when the expression level of Olig2 is 0.0068- to 0.0084-fold of that of GAPDH on 7 to 8 days of culture of the embryoid body. In still another embodiment of the present invention, the stem cell is a stem cell forming an embryoid body (EB) whose pluripotency and differentiation ability increase when the expression level of T is 0.051- to 0.064-fold of that of GAPDH on 7 to 8 days of culture of the embryoid body. In still another embodiment of the present invention, the stem cell is a stem cell formming an embryoid body (EB) whose pluripotency and differentiation ability increase when the expression level of Nkx2.5 is 0.086- to 0.110-fold of that of GAPDH on 7 to 8 days of culture of the embryoid body. In still another embodiment of the present invention, the stem cell is a stem cell forming an embryoid body (EB) whose pluripotency and differentiation ability increase when the expression level of Oct4 is 0.050- to 0.061-fold of that of GAPDH on 7 to 8 days of culture of the embryoid body. In still another embodiment of the present invention, the stem cell is a stem cell forming an embryoid body (EB) whose pluripotency and differentiation ability increase when the expression level of Esrrbb is 0.012- to 0.016-fold of that of GAPDH on 7 to 8 days of culture of the embryoid body. In still another embodiment of the present invention, the stem cell is a stem cell forming an embryoid body (EB) whose pluripotency and differentiation ability increase when tp the expression level of Esrrbb is 0.41- to 0.52-fold of that of GAPDH on 7 to 8 days of culture of the embryoid body. In still another embodiment of the present invention, the stem cell is a stem cell forming an embryoid body (EB) whose pluripotency and differentiation ability increase when the expression level of Klf4 is 0.071- to 0.088-fold of that of GAPDH on 7 to 8 days of culture of the embryoid body. In still another embodiment of the present invention, the stem cell is a stem cell forming an embryoid body (EB) whose pluripotency and differentiation ability increase when the expression level of cTnT is 2.10- to 2.58-fold of that of GAPDH on 7 to 8 days of culture of the embryoid body. In still another embodiment of the present invention, the stem cell is a stem cell forming an embryoid body (EB) whose pluripotency and differentiation ability increase when the expression level of a-Actin is 3.79- to 4.65-fold of that of GAPDH on 7 to 8 days of culture of the embryoid body. In still another embodiment of the present invention, the stem cell is a stem cell forming an embryoid body (EB) whose pluripotency and differentiation ability increase when the expression level of Mlc2v is 10.70- to 13.11-fold of that of GAPDH on 7 to 8 days of culture of the embryoid body. In still another embodiment of the present invention, the stem cell is a stem cell forming an embryoid body (EB) whose pluripotency and differentiation ability increase when the expression level of Runx1 is 0.16- to 0.21-fold of that of GAPDH on 7 to 8 days of culture of the embryoid body.

To achieve the above objects, the present invention provides a pharmaceutical composition for treating asthma, comprising, as an active ingredient, the therapeutic stem cell composition as described above.

To achieve the above objects, the present invention provides a pharmaceutical composition for treating allergic asthma, comprising, as an active ingredient, the therapeutic stem cell composition as described above.

Hereinafter, the present invention will be described in further detail with reference to examples. However, these examples are provided to help understanding of the present invention, and the scope of the present invention is not limited to these examples.

### Advantageous Effects

Therapeutic stem cells which are provided according to the present invention are effective in that they have an identified specific gene expression ratio enabling good-quality stem cells to be screened and the necessary characteristics of the stem cells can be selectively managed.

### DESCRIPTION OF DRAWINGS

FIG. 1 is a graph showing intracellular glutathione (GSH) levels that modulate the self-renewal and migration activities of mesenchymal stem cells. It shows luminescence-based quantification of GSH in cell lysates (n=2 independent biological replicates) following treatment with 100 μM H₂O₂ (n=3 cells) in FR^{High}, FR^{Mid}, and FR^{Low} hES-MSCs.

FIGS. 2A and 2B show luminescence-based quantification based on comparison of fluorescence.

FIG. 3 shows the results of measuring the difference in differentiation activity-related gene expression between stem cells sorted by a FreSH-tracer. ns: not significant, ***p<0.001.

FIGS. 4A, 4B, 4C and 4D are graphs showing analyses of colony-forming unit fibroblasts (CFU-F; n=15), limiting dilution by replating primary CFU colonies (n=6), chemotaxis to stromal derived factor-1α (SDFα; 150 ng/mL, n=8), and chemotaxis to 10 ng/mL platelet-derived growth factor (PDGF)-AA in the absence or presence of STI571 (0.5 μg/mL), a PDGFR inhibitor (n=8).

FIG. 5 is a set of graphs showing the results of qPCR of pluripotency-related genes (n=8) in hES-MSCs sorted based on the FR and in unsorted control (naive) cells.

FIG. 6 is a set of graphs showing the results of qPCR of cell migration-related genes (n=8) in hES-MSCs sorted based on the FR and in unsorted control (naive) cells.

FIG. 7 is a set of graphs showing the results of measuring the difference in expression of stem cell growth factors and their receptor genes in hES-MSCs sorted based on the FR and in unsorted control (naive) cells.

FIG. 8 shows the results of measuring the difference in expression of immunomodulatory and anti-inflammatory genes in stem cells sorted by a FreSH-tracer.

FIGS. 9A and 9B are graphs showing the functional role of high GSH levels in hES-MSCs (CFU-F (n=10)).

FIG. 10 is a set of graphs showing qPCR assays of stemness and migration-related genes (n 32 4) in control and BSO-treated FR^{High}hES-MSCs or control and GSH-EE supplemented FR^{Low} cells. Scale bars, 200 μm. For all bar graphs, values represent mean±SEM; *p<0.05, **p<0.01, ***p<0.001; n.s., not significant.

FIG. 11A is a graph showing analysis of colony-forming unit fibroblasts (CFU-F) and FIG. 11B is a graph showing analysis of chemotaxis to 10 ng/mL platelet-derived growth factor (PDGF)-AA, in BSO- and GSH-EE-treated FR^{High} and FR^{Low} hES-MSCs.

FIGS. 12A and 12B show analysis of 10 ng/mL PDGF-AA (n=10) in unsorted control (naive) cells treated with BSO or GSH-EE. Quantitative data are represented as the ratio to non-treated naive cells

FIG. 13 is a set of graphs showing impaired pluripotency and differentiation in murine embryonic stem cells with low glutathione (GSH) levels. It shows luminescence-based quantification of GSH in cell lysates (n=4).

FIG. 14 is a graph showing clonogenic capacity in limiting dilution (n=5) in FR^{High} and FR^{Low} mESCs and in unsorted control (naive) cells. Scale bar, 200 μm.

FIG. 15A is a graph showing qPCR and FIG. 15B is an annotated image showing western blot analyses (n=4), in FR^{High} and FR^{Low} mESCs and in unsorted control (naive) cells. Scale bar, 20 μm.

FIG. 16 is a graph showing qPCR analyses for lineage-specific genes (Neurog2, Olig2, T, and Nkx2.5) in EBs at the indicated day (n=4).

FIG. 17 is a graph showing that mESC sorted by FR into two populations (FR^{High} and FR^{Low} cells) were differentiated by forming embryoid body (EB). qPCR analyses (n=4) of the pluripotency and lineage-specific genes in cells from EB were performed at the indicated day. Quantitative data are represented as the ratio to FR^{High} cells of day 0. All error bars represent the mean±SEM. *P<0.05, **P<0.01, ***P<0.001, one- or two-way ANOVA with Bonferroni post-hoc tests. Scale bar, 200 μm.

FIG. 18 is a graph showing qPCR analyses of ρIII-tubulin+ neuron cells of neural lineage markers (Neurog1, Neurod1, Nkx2.2, Ascl2, fap, S100b, and Olig2; n=4) during ESC differentiation. Scale bars, 100 μm. Values represent mean±SEM; *p<0.05, **p<0.01, ***p<0.001.

FIG. 19 is a scheme for observing the increased therapeutic efficacy of stem cells with high glutathione levels.

FIG. 20 shows the results of administering naive, FR^{High}, and FR^{Low} hES-MSCs or PBS to mice treated with ovalbumin and poly(I:C).

FIG.L 21 is a graph showing the number of total cells, macrophages, neutrophils, lymphocytes, and eosinophils (n=30).

FIG. 22 is a graph showing ELISA-based detection of TNFα, IL-10, and IL-17 in the bronchoalveolar lavage fluid (n=10).

FIG. 23 is a graph showing qPCR analysis of Tnfa, Clcl2, Il1b, Il12a, and Il18 expression using RNA isolated from the lung tissues (n=10). Scale bar, 200 μm. For all bar graphs, values represent mean±SEM; *p<0.05, **p<0.01, ***p<0.001; ns., not significant.

### BEST MODE

Therapeutic stem cells which are provided according to the present invention are those obtained by sorting according to their characteristics based on an identified specific gene expression ratio such that the necessary characteristics thereof can be selectively managed. The present invention provides a therapeutic stem cell composition comprising a stem cell in which the expression level of any one or more gene selected from the group consisting of ACAN, SOX9, AP2, RUNX2, OCN, ALP, OCT4, SOX2, CXCR4, cMET, PDGFRA, PDGFRB, VEGF-R1, VEGF-R2, CSF-1, IDO2, Sox2, Nanog, cMyc, Klf2, Klf4, Rex1, Esrrb, Neurog1, Neurod1, Nkx2.2, Ascl2, Gfap, S100b, Olig2, Neurog2, T, Nkx2.5, Esrrbb, Klf2, Klf4, cTnT, a-Actin, Mlc2v, and Runx1, is higher or lower than the expression level of GAPDH (glyceraldehyde 3-phosphate dehydrogenase) gene.

### MODE FOR INVENTION

Hereinafter, the present invention will be described in detail with reference to examples. However, these examples are for illustrative purposes only and the scope of the present invention is not limited by these examples.

### FreSH-Tracer-based Measurement of Antioxidant Activity of Stem Cells

Method for Measurement of Stem Cells

The present inventors have identified the relationship between the fluorescence intensity ratio of the FreSH-tracer (Fluorescent Real-time SH group-tracer) of the present invention in a certain wavelength range and the antioxidant activity of a sample, and have found that the antioxidant activity of cells can be monitored in real time by observing the above-described fluorescence intensity, thereby completing the present invention

A method for measuring the antioxidant activity (or anti-oxidative activity) of stem cells comprises: (a) brining a composition for measuring cell activity, which comprises a compound represented by the following formula 1 or a salt thereof, into contact with cells; and (b) observing in real-time, for example, (i) the fluorescence intensity at 510 nm or 580 nm in the cells or (ii) the ratio of the fluorescence intensity at 510 nm to the fluorescence intensity at 580 nm in the cells:

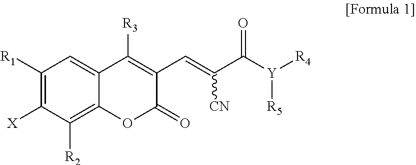

In this Example, a compound represented by formula 2 was preferably used. The compound represented by formula 2 was used at a concentration of 1 to 100 μM, preferably 1 to 20 μM, more preferably 1 to 10 μM, even more preferably about 5 μM.

GSH Levels Different between Cellular Organelles of Living Cells

To utilize a FreSH-tracer for GSH monitoring in living cells, its cytotoxicity was evaluated. Treatment of up to 10 μM FreSH-tracer for 24 hours showed no effect on the viability of HeLa cells, human bone marrow-derived mesenchymal stem cells (hBM-MSCs) and human embryonic stem cell-derived mesenchymal stem cells (hES-MSCs). HeLa cells were equilibrated with treatment of 5 μM FreSH-tracer for 2 hours. Confocal and ratiometric pseudo-color images revealed that the FreSH-tracer was distributed inside the cells, exhibiting a wide range of FR values. The FR in the nucleus was about 1.5- to 2-fold higher than that of the cytoplasm. The FR of the nucleolus revealed relatively lower GSH levels, and the FR of the peripheral cytoplasm was higher than that of other regions. Moreover, variable FR values were observed in the cytoplasm, which produced a mosaic pattern in the pseudo-color images, possibly arising from GSH in the ER and mitochondria. When HeLa cells were treated with diamide following equilibration with 5 μM FreSH-tracer for 2 hours, the FR gradually decreased by diamide (or NEM) and then rapidly increased by DTT. This suggests that the FreSH-tracer reacts reversibly with thiols in the intracellular environment.

The mitochondrion is the major site of endogenous ROS generation during normal oxidative metabolism. Cytosolic GSH is transported to the mitochondria, preventing macromolecular damage and modulating ROS-induced signaling. To further analyze the FR heterogeneity in the cytoplasm, a mitochondria-targeting FreSH-tracer derivative, designated MitoFreSH-tracer was synthesized by attaching a triphenylphosphonium moiety to a FreSH-tracer. The MitoFreSH-tracer reacted rapidly and reversibly with GSH, exhibited GSH-dependent FR values similar to those of the FreSH-tracer (KD=1.3 mM). It showed no cytotoxic effect on HeLa cells with treatment up to 10 μM for 24 hours. Confocal images revealed that the MitoFreSH-tracer localized to the mitochondria in HeLa cells, and the FR decreased upon diamide treatment. This indicates that GSH levels within the mitochondria can be monitored by the MitoFreSH-tracer. Remarkably, there was wide variation in the FR values among mitochondria within a single cell, even under normal culture conditions, indicating that the GSH levels in the mitochondria of a single cell are heterogeneous. Moreover, a concentration-dependent decrease in the FR was observed when the cells were treated with antimycin A, which generates ROS in mitochondria by inhibiting electron transport. Simultaneous analyses showed that mitochondrial ROS levels increased with treatment of DHR123, a non-fluorescent rhodamine derivative that localizes to the mitochondria and emits fluorescence when oxidized by ROS. Taken together, these results demonstrate that GSH levels differ among organelles as well as among different regions within the same compartment of a live cell.

Real-Time Measurement of GSH Concentrations in Living Cells

Although GSH is the most abundant thiol in cells, proteins constitute a significant portion of cellular thiol. Thus, the present inventors examined whether the FreSH-tracer could be not significantly affected by the presence of protein thiols for reporting the continuous changes of GSH levels in living cells. When HeLa cells were treated with various concentrations of buthionine sulfoximine (BSO) for 48 hours to suppress GSH synthesis, measurements of the FR in the GSH-depleted cells showed that the FR_{GSH} accounted for approximately 55% of the total FR in normal cells, and the intracellular GSH concentration, which was independently measured by luminescence-based assays in cell lysates, was directly correlated with the FR determined by confocal microscopy (R²=0.9135) and flow cytometry (R²=0.9753).

The in vitro experiments described above established that H₂O₂ treatment diminishes only the FR_{GSH}, while having little effect on the FR_{PSH}. In line with these in vitro data, the GSH-depleted cells showed no change in their FR values following the addition of either 100 μM or 500 μM H₂O₂ over a period of 40 minutes, indicating that oxidation of GSH, and not PSH (cysteine thiols in cellular proteins), caused the FR change in H₂O₂-treated cells. Thus, the FreSH-tracer can report the real-time dynamic changes of GSH concentration in live cells under oxidative stress. When the GSH-depleted cells were treated with diamide as a control experiment, the FR decreased but was then immediately restored to the original level. This restoring activity was abrogated by treatment with 1-chloro-2,4-dinitrobenzene, an inhibitor of thioredoxin reductase, indicating that thioredoxin, instead of GSH, is required to reduce the disulfides of PSH. These results indicate that the FreSH-tracer can successfully distinguish between GSH and PSH in living cells.

Cellular GSH Levels That Dynamically Change under Oxidative Stress

ROS production by various cellular conditions significantly affected stem cell functions such as self-renewal and differentiation. Thus, the present inventors monitored the H₂O₂-induced changes in GSH levels. When HeLa cells and hBM-MSCs were treated with H₂O₂, the FR decreased rapidly, then remained unchanged before increasing slowly, and ultimately returned to the untreated level. The profile and time course of FR changes in the cytoplasm and nucleoplasm were similar to those observed in whole cells. Notably, GSH levels in HeLa cells were more sensitive to H₂O₂ treatment than those in hBM-MSCs. In HeLa cells treated with increasing concentrations of H₂O₂, both the decrease in the FR and the lag time for recovery were accentuated.

To confirm these results, the present inventors monitored the GSH changes induced by endogenously produced ROS. In macrophages, ROS are produced by NADPH oxidase when the cells are activated. Therefore, RAW264.7 cells were loaded with the FreSH-tracer and treated with phorbol 12-myristate 13-acetate (PMA). Confocal microscopy revealed that the FR decreased gradually over 30 minutes upon PMA treatment in every region of the treated cells and was then slowly restored to the control level during the following 30 minutes. Moreover, ROS production was also reported to increase in cells cultured at a low density or in serumdeprived medium. Therefore, the present inventors monitored the effect of culture conditions on the changes of GSH levels. Exposure of HeLa cells to serum-free medium for 18 hours significantly reduced the FR in the cytoplasm and in the nucleoplasm. When cultured at different densities, the average FR of densely cultured HeLa cells was significantly higher than that of sparsely cultured cells, despite the large variation in the FR, particularly in the nucleoplasm.

To monitor the GSH levels in hBM-MSCs, the cells were serially subcultured at different seeding densities. Flow-cytometric analysis showed that the hBM-MSC populations were heterogeneous with respect to GSH levels, and, notably, the number of cells with a high GSH content (GSH^{High}) gradually decreased with increasing passages, especially when cultured at a low cell density, indicating that GSH levels in stem cells depend on the culture conditions. These results demonstrate that GSH levels are dynamically changed in response to oxidative stress, and that the FreSH-tracer can provide spatiotemporal information of GSH levels for estimating the redox buffering capacity of individual cells.

GSH Levels for Stem Cell Function

To further explore the biological significance of the reprogrammed GSH levels in stem cells, the present inventors sorted hBM-MSCs (human bone marrow-derived mesenchymal stem cells) by flow cytometry and divided them into three subpopulations based on the FR (FR^{High}, FR^{Mid}, and FR^{Low} cells; FIG. S5A). Then, the FreSH-tracer in sorted cells was rapidly removed. The FR^{High} hBM-MSCs, compared with FR^{Mid} and FR^{Low} cells, significantly enhanced the cellular functions regarding colony-forming unit fibroblasts (CFU-F) and the chemoattraction to platelet-derived growth factor (PDGF).

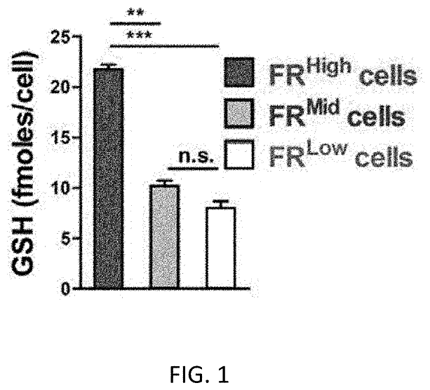

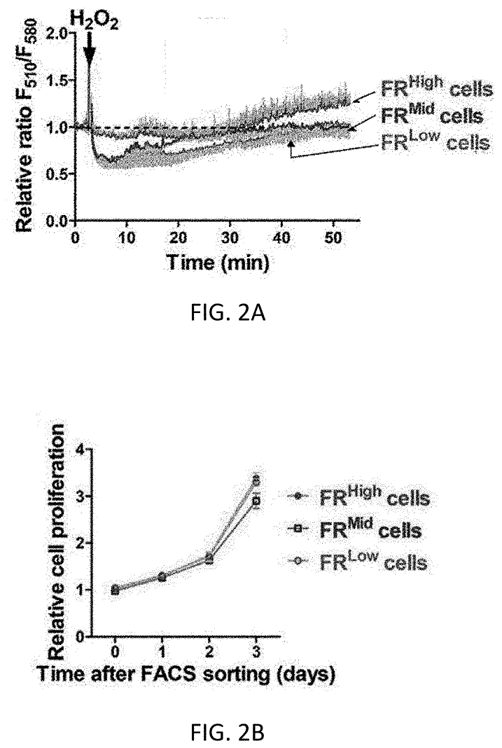

To validate the improved functionality of stem cells with a high GSH content, the present inventors sorted hES-MSCs into FR^{High}, FR^{Mid}, and FR^{Low} subpopulations based on the FR The GSH concentrations in the cell lysates from each population were directly proportional to their FR levels, validating the FR-based sorting method (FIG. 1). Interestingly, the decrease of FR and the lag time for recovery following treatment with 100 μM H₂O₂ were inversely proportional to the FR levels of the sorted cell population (FIG. 2A). Moreover, the FR of FR^{High} cells recovered to higher than basal levels after H₂O₂ exposure, indicating that FR cells have greater GSH-restoring capacity compared with control cells.

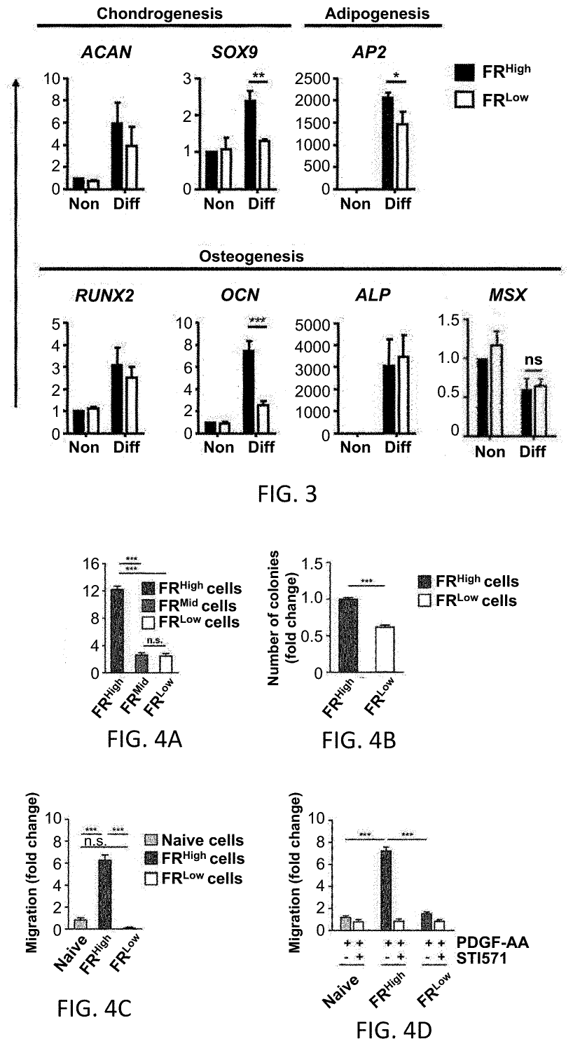

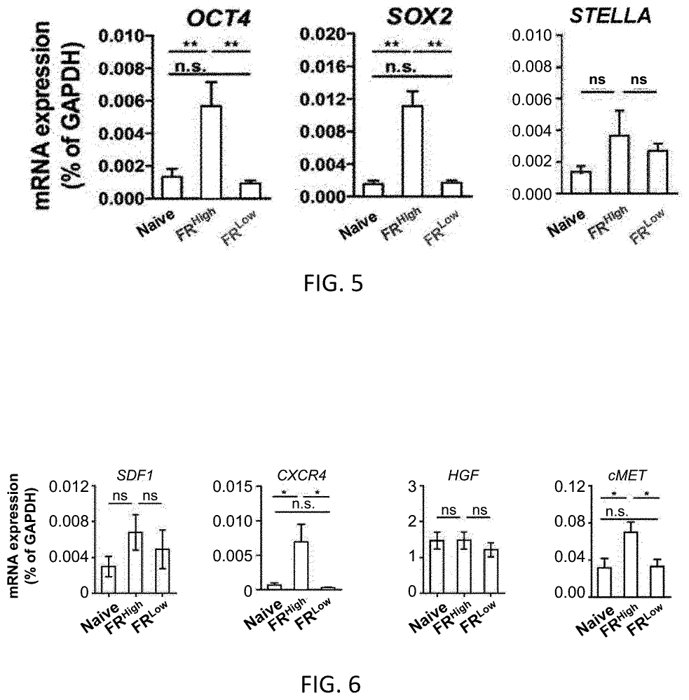

As shown in FIG. 2B, the sorted hES-MSCs showed no significant difference in proliferation rate. When multipotency was examined, both FR^{High} and FR^{Low} cells exhibited a similar capacity to differentiate into chondrogenic, adipogenic, and osteogenic lineages. However, FR^{High} hES-MSCs, compared with FR^{Low} cells, showed a significant increase in the induction of some lineage markers, including SOX9, AP2, and OCN (FIG. 3). At this time, for differentiation media, the cells were maintained and cultured in each of adipogenic induction medium (DMEM supplemented with 5% FBS, 1 mM dexamethasone, 10 mM insulin, 200 mM indomethacin and 0.5 mM isobutylmethylxanthine), osteogenic induction medium (DMEM supplemented with 5% FBS, 50 mM ascorbate-2-phosphate, 0.1 mM dexamethasone, and 10 mM glycerophosphate), and chondrogenic induction medium (e.g., StemPro chondrogenesis, Invitrogen). FIG. 3 is a graph showing the results of qPCR performed to quantify lineage-specific genes in non-differentiated(Non) FR^{High} cells and FR^{Low} cells and differentiated(Diff) FR^{High} cells and FR^{Low} cells. Additionally, FR^{High} hES-MSCs had approximately 4.7- and 4.9-fold higher numbers of CFU-F than did FR^{Mild} and FR^{Low} cells, respectively (FIG. 4A). When individual CFU colonies were harvested and reseeded for limiting dilution assay, CFU colonies from FR^{High} hES-MSCs showed two times the clonogenic activity than those from FR^{Low} cells (FIG. 4B), indicating the enhanced self-renewal activity of FR^{High} hES-MSCs. FR^{High} cells in both types of stem cells showed significantly enhanced chemoattraction to stromal derived factor-1 compared with naive or FRFR^{Low} cells (FIG. 4C). The improved chemotactic activities in FR^{High} cells were also found by PDGF stimuli and were significantly blocked by a PDGF receptor (PDGFR) inhibitor, STI571 (FIG. 4D). Accordingly, FR^{High} hES-MSCs showed significantly higher mRNA levels of pluripotency- or migration-related genes than did naive and FR^{Low} cells, including OCT4 and CXCR4 (see FIGS. 5 to 8).

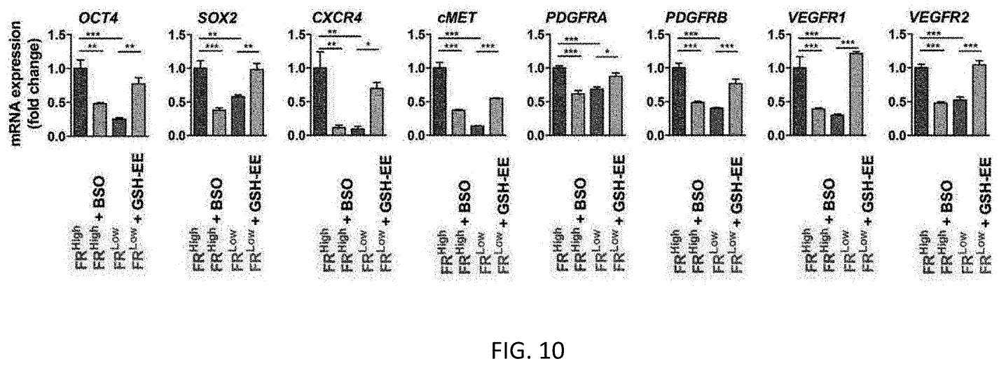

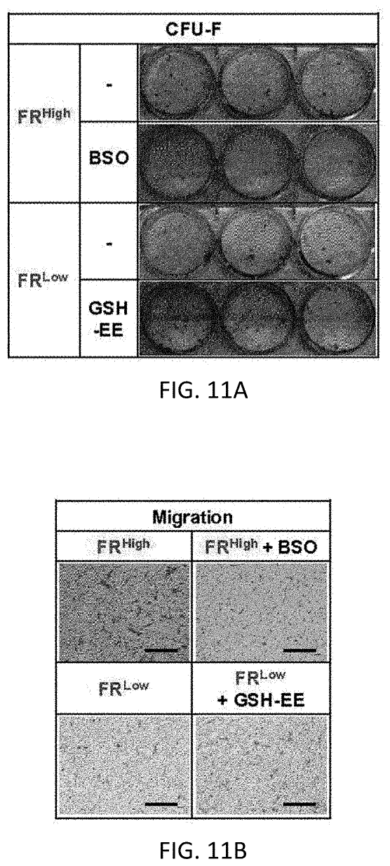

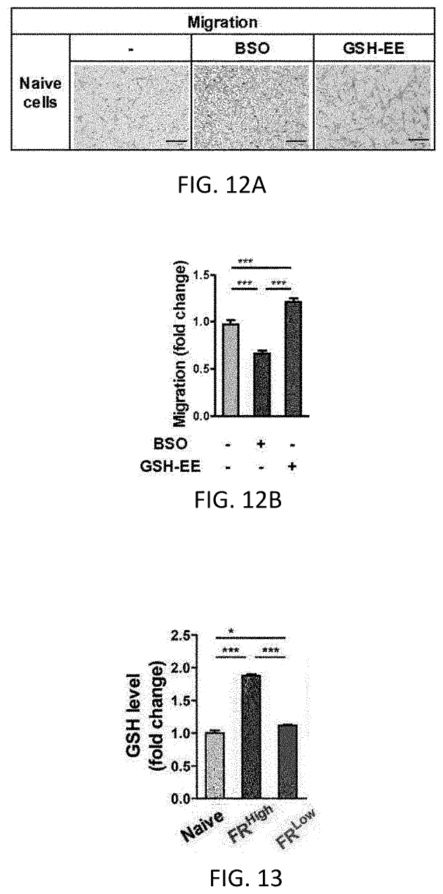

To prove the functional role of high GSH levels, the present inventors depleted cellular GSH in FR^{High} hES-MSCs using buthionine sulfoximine (BSO), and found that GSH depletion severely impaired the enhanced clonogenic and migration capacities as well as upregulation of the related genes observed in FR^{High} hES-MSCs (FIGS. 9A, 9B, 10, 11A and 11B). In line with these data, declined cellular functions in FR^{Low} hES-MSCs were reversed to levels similar to those of FR cells by glutathione ethyl ester (GSH-EE), a cell-permeable glutathione. Moreover, naive cells treated with BSO and GSH-EE showed the significant repression and activation of chemoattraction to PDGF, respectively (FIGS. 12A and 12B).

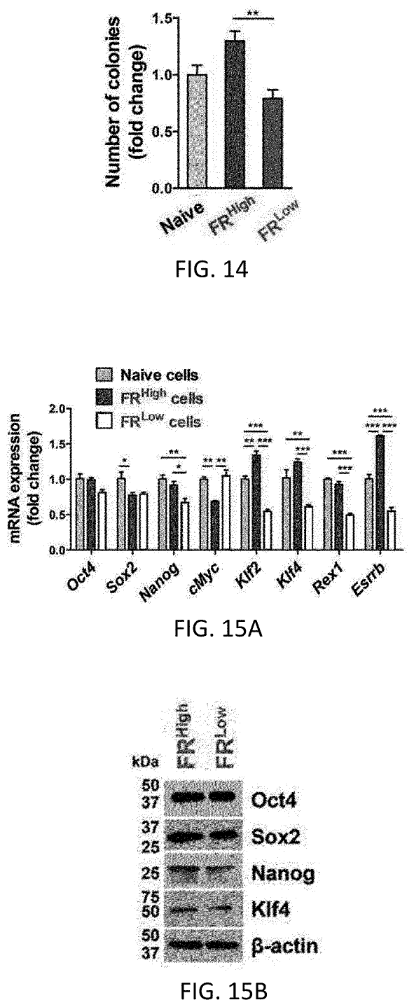

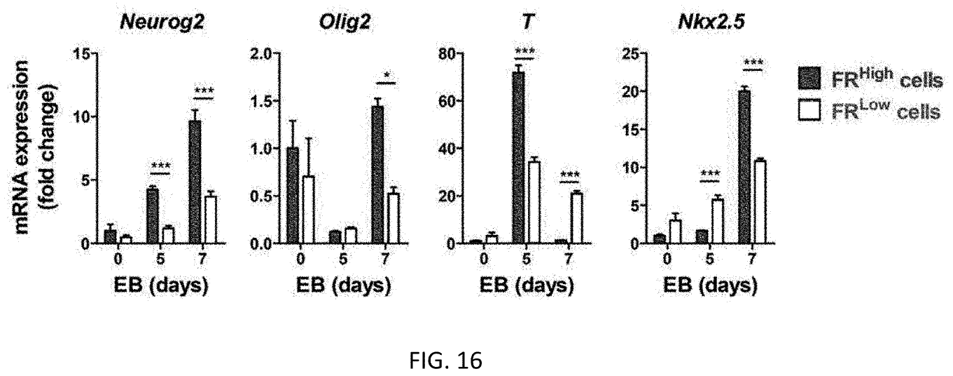

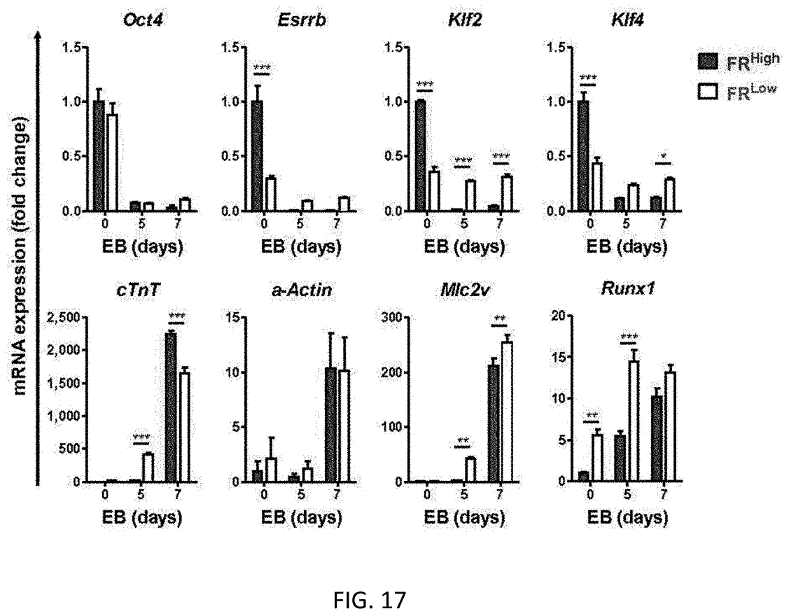

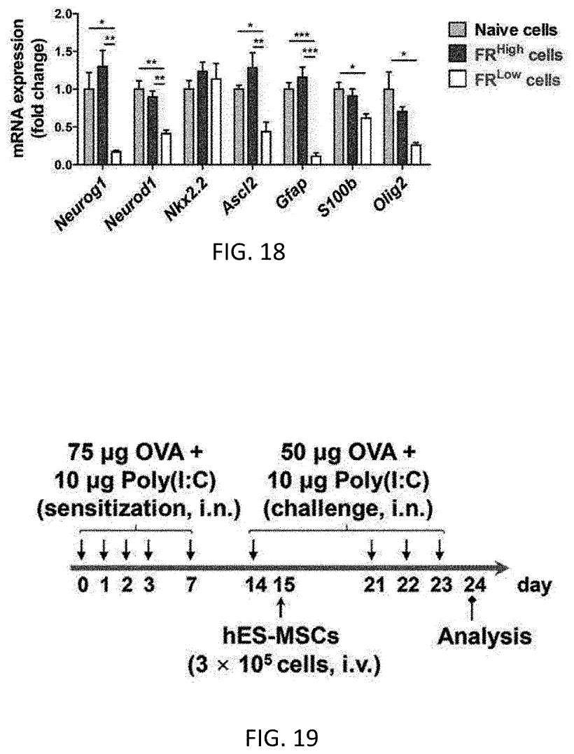

To investigate the significance of a high GSH content among other types of stem cells, the present inventors fractionated murine embryonic stem cells (mESCs) into higher- and lower-GSH level cells based on the FR of the FreSH-tracer (FIG. 13). Compared with FR^{Low} mESCs, FR^{High} cells displayed remarkably enhanced cellular function regarding GSH recovery capacity following H₂O₂ treatment. These FRFR^{High} cells exhibited dome-like morphological colonies with positive alkaline phosphatase staining, characteristic of undifferentiated embryonic stem cells. In addition, FR^{High} mESCs showed representing self-renewal activity (FIG. 14) and greater expression of pluripotency-related genes (FIGS. 15a and 15b) as exhibited in the limiting dilution assay. Thus, FR^{High} mESCs were superior to FR^{Low} cells in terms of clonogenic efficiency. When they were differentiated by forming embryoid bodies (EBs), FRFR^{Low} mESCs exhibited defects in EB formation and induction of several lineage markers, such as neural (e.g., Neurog2 and Olig2) and mesodermal (e.g., T and Nkx2.5) markers (FIGS. 16 and 17). The defective differentiation capacity in cells from FR^{Low} embryoid bodies was further validated by in vitro neuronal differentiation, and evidenced by a lack of β III-tubulin⁺ neurons and by impaired induction of neuronal markers (FIG. 18). Taken together, these findings demonstrate that high cellular GSH levels are required for maintaining the core functions in stem cells.

### Analysis of Gene Expression in Cells Sorted Based on Fresh-Tracer

i) FreSH-Tracer-Based Analysis in Mesenchymal Stem Cells (hES-MSCs)

Mesenchymal stem cells (hES-MSCs) were sorted based on the FR of the FreSH-tracer, and the genetic difference between the FR^{High} and FR^{Low} cell subpopulations was analyzed. First, at 2 weeks after induction of differentiation, changes in expression of RUNX2, OCN, ALP and MSX, genes associated with osteogenic differentiation ability, were measured by RQ-PCR Total RNA (50 ng) was reverse-transcribed using Taqman reverse transcription reagent (Applied Biosystems, Canada), and the threshold cycle (Ct) was determined using RQ-PCR as known in the art. The relative expression levels of the target genes were determined using the 2^{-ΔΔ}^{_{Ct}} method, and GAPDH was used as an endogenous control gene. As a result, as shown in FIG. 3, it was confirmed that the expression of OCN was significantly higher in FR^{high} cells than in the FR^{low} cells.

Meanwhile, the relative expression levels of osteogenic differentiation-related genes in FR^{high} compared to those in FR^{low} cells were measured. It was observed that there was no difference in gene expression level of ATF2, HEY1, FOSL1, FOSL2, FSHB, JUN, JUNB, JUND, GRB2, KITLG, MITF, and OLR1 genes. However, as shown in Table 1 below, it was observed that the relative expression levels of RUNX2 and OCN genes were 1.22-fold and 2.96-fold higher, respectively, in FR^{high} cells than in FR^{low} cells. Moreover, it was observed that the relative expression level of ALP gene was 0.87-fold lower in FR^{high} cells.

In addition, as shown in FIG. 3, the relative expression levels of chondrogenic differentiation-related genes (ACAN and SOX9) were 1.52-fold and 1.83-fold higher, respectively, in FR^{high} cells than in FR^{low} cells. Measurement of the relative expression levels of adipogenic differentiation-related genes indicated that the expression level of AP2 was 1.22-fold higher in FR^{high} cells than in FR^{low} cells.

TABLE 1

| | | | | Relative mRNA |
|---|---|---|---|---|
| | | | | expression level |
| | | mRNA expression level | | (FR^{High} to |
| Cell activity | Expressed | (% of GAPDH) | | FR^{Low}) after |
| (hES-MES) | genes | FR^{High} cells | FR^{Low} cells | differentiation |
| Chondrogenic | ACAN | 0.00109 | 0.005188 | 1.52:1 |
| differentiation | SOX9 | 0.00697 | 0.003841 | 1.83:1 |
| Adipogenic | AP2 | 10.36347 | 8.049051 | 1.22:1 |
| differentiation | | | | |
| Osteogenic | RUNX2 | 0.432153 | 0.353545 | 1.22:1 |
| differentiation | OCN | 0.083049 | 0.004915 | 2.96:1 |
| | ALP | 30.68782 | 45.24346 | 0.87:1 |

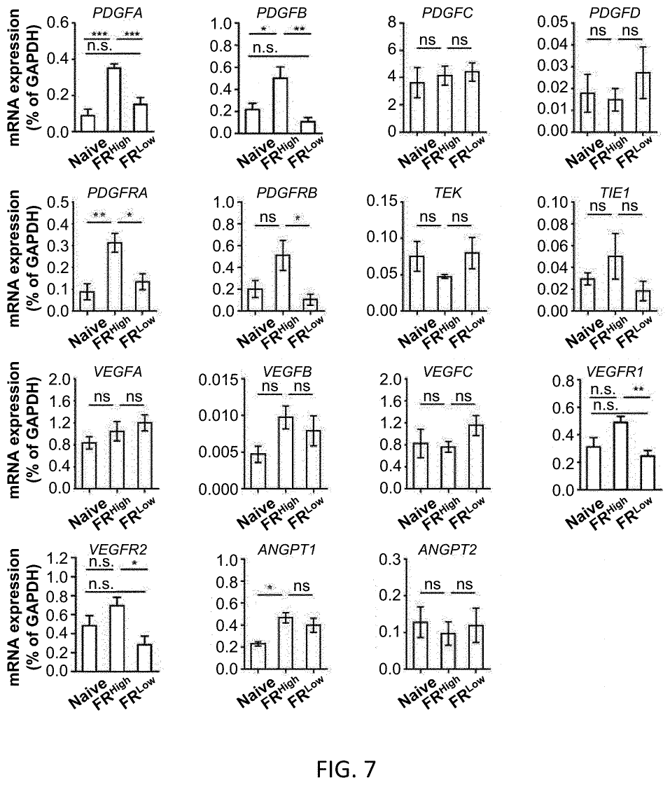

In addition, changes in expression of genes related to pluripotency, cell migration, mesenchymal stem cell therapeutic efficacy, growth factor and growth factor receptor, anti-inflammation and immunomodulation, were analyzed by qPCR As a result, it was confirmed that FR^{high} cells showed a significant increase in the expression of pluripotency-related genes (OCT4, SOX2 and CXCR4) and HGF receptor (cMET) compared to naïve cells (FIGS. 5 and 6), and also showed a significant increase in the expression of the PDGF receptors (PDGF-RA and PDGF-RB) and VEGF growth factor receptors (VEGFR1, VEGF-R2 and ANGPT1) related to the in vivo engraftment rate, viability and vascular regeneration ability of MSCs (FIG. 7).

Specifically, the relative expression levels of pluripotency-related genes in FR^{high} cells compared with those in naïve cells were measured. It was observed that there was no difference in gene expression level of ATF2, HEY1, FOSL1, FOSL2, FSHB, JUN, JUNB, JUND, GRB2, MUG, MITF and OLR1 genes. However, as shown in FIG. 5, it was confirmed that the relative expression levels of OCT4 and SOX2 genes were 4.3-fold and 7.2-fold higher, respectively, in FR^{high} cells than in naïve cells. In addition, as shown in FIG. 6, measurement of the relative expression levels of migration-related genes indicated that the expression level of CXCR4 was 10.2-fold higher in FR^{high} cells than in naïve cells (Table 2).

TABLE 2

| | | | | | Relative |
|---|---|---|---|---|---|
| | | | | | mRNA |
| | | | | | expression |
| | | | | | level (FR^{High} |
| Cell activity (hES- | Expressed | mRNA expression level (% of GAPDH) | | | to input |
| MES) | genes | Input cells | FR^{High} cells | FR^{Low} cells | (naïve)) |
| Phuipotency | OCT4 | 0.001329 | 0.00567 | 0.000919 | 4.3:1 |
| | SOX2 | 0.001533 | 0.01108 | 0.001672 | 7.2:1 |
| Migration | CXCR4 | 0.000683 | 0.006942 | 0.000281 | 10.2:1 |
| Growth and | cMET | 0.031626 | 0.070111 | 0.03313 | 2.2:1 |
| proliferation | | | | | |
| Engraftment rate, | PDGFRA | 0.088231 | 0.351605 | 0.151377 | 4.0:1 |
| viability and | PDGFRB | 0.218623 | 0.501705 | 0.107756 | 2.3:1 |
| vascular | VEGF-R1 | 0.31058 | 0.491561 | 0.246589 | 1.6:1 |
| regeneration | VEGF-R2 | 0.483668 | 0.694414 | 0.283608 | 1.4:1 |
| Immunomodulation | CSF-1 | 0.083195 | 0.212191 | 0.104215 | 2:1 |
| Anti-inflammation | IDO2 | 0.000128 | 0.001454 | 0.000167 | 10:1 |

Meanwhile, as shown in FIG. 6, the relative expression levels of growth- and proliferation-related genes in FR^{high} cells compared with those in naïve cells were measured. It was observed that there was no difference in gene expression levels of ATF2, HEY1, FOSL1, FOSL2, FSHB, JUN, JUNB, JUND, GRB2, KITLG, MITF, and OLR1 genes. However, it was confirmed that the relative expression level of cMET gene was 2.2-fold higher in FR' cells than in naïve cells (Table 2).

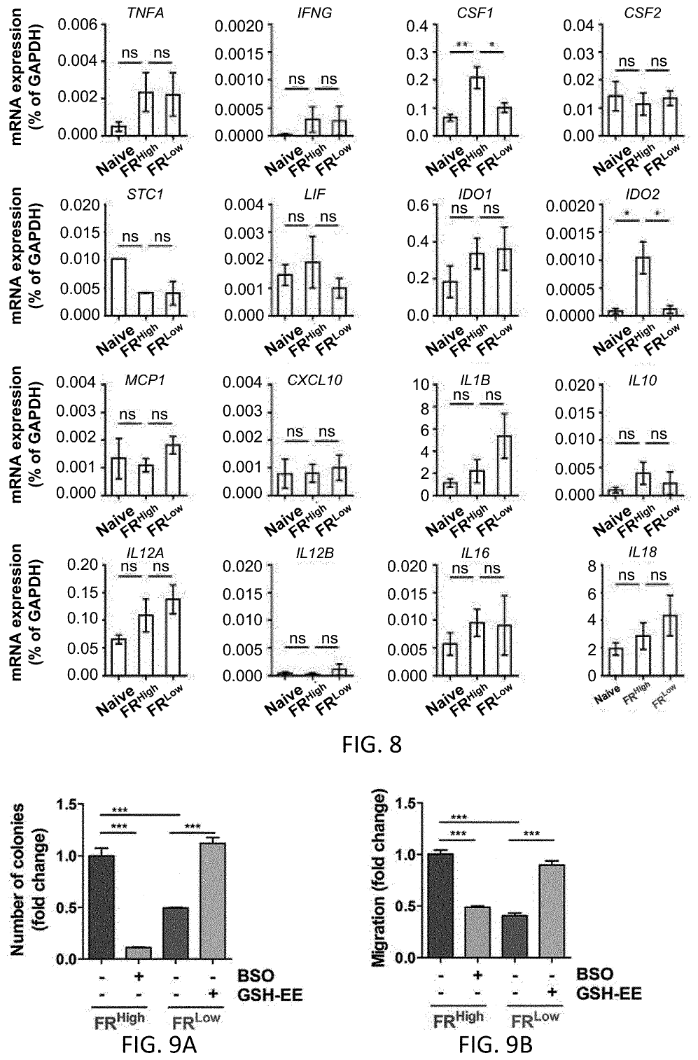

In addition, as shown in FIG. 8, it was confirmed that the expression levels of CSF-1 (macrophagecolony stimulating factor-1), related to the immunomodulatory activity of mesenchymal stem cells (MSCs), and the representative anti-inflammatory gene IDO2 (indoleamine 2,3-dioxygeniase 2), significantly increased in FR^{high} cells (see FIG. 8 and Table 2).

Similarly, the relative expression levels of genes, related to engraftment rate, viability and vascular regeneration, in FR cells compared with those in naïve cells, were measured. It was observed that there was no change in gene expression level of ATF2, HEY1, FOSL1, FOSL2, FSHB, JUN, JUNB, JUND, GRB2, KITLG, MITF, and OLR1 genes. However, as shown in FIG. 7 and Table 2 above, the relative expression levels of PDGFRA, PDGFRB, VEGF-R1 and VEGF-R2 genes were 4.0-fold, 2.3-fold, 1.6-fold and 1.4-fold higher, respectively, in FR^{high} cells than in naïve cells.

In addition, the relative expression levels of immunomodulation-related genes in FR^{high} compared with those in naïve cells were measured. It was observed that there was no difference in gene expression level of ATF2, HEY1, FOSL1, FOSL2, FSHB, JUN, JUNB, JUND, GRB2, KITLG, MITF and OLR1 genes. However, it was confirmed that the relative expression level of CSF-1 gene was 2-fold higher in FR^{high} cells than in naïve cells.

Furthermore, the relative expression levels of anti-inflammation-related genes in FR^{high} cells compared with those in naïve cells were measured. It was observed that there was no difference in gene expression level of ATF2, HEY1, FOSL1, FOSL2, FSHB, JUN, JUNB, JUND, GRB2, KITLG, MITF and OLR1 genes. However, it was confirmed that the relative expression level of IDO2 gene was 10-fold higher in FR^{high} cells than in naïve cells.

ii) FreSH-Tracer-Based Analysis in Embryonic Stem Cells (mESCs)

Embryonic stem cells (mESC) were sorted based on the FR of the FreSH-tracer, and the genetic difference between the FR^{high} and FR^{low} cell subpopulations was analyzed in the same manner as described. The relative expression levels of pluripotency-related genes in FR^{high} cells compared with those in FR^{low} cells were measured. As shown in Table 3 below and in FIGS. 15A and 15B, it was confirmed that the relative expression levels of Oct4, Nanog, Klf2, Klf4, Rex1 and Esrrb genes were 1.22-fold, 1.36-fold, 2.46-fold, 2.02-fold, 1.88-fold and 2.92-fold higher, respectively, in FR^{high} cells than in FR^{low} cells. Furthermore, it was confirmed that the relative expression levels of Sox2 and cMyc were 0.98-fold and 0.65-fold lower, respectively, in FR^{high} cells.

Meanwhile, the relative expression levels of neuronal differentiation promotion-related genes in FR^{high} cells compared with those in FR^{low} cells were measured. As shown in Table 3 below and FIG. 18, the relative expression levels of Neurog1, Neurod1, Nkx2.2, Ascl2, Gfap, S100b and Olig2 genes were 7.56-fold, 2.16-fold, 1.09-fold, 2.91-fold, 10.42-fold, 1.48-fold and 2.71-fold higher, respectively, in FR^{high} cells than in FR^{low} cells.

TABLE 3

| | | | | | Relative |
|---|---|---|---|---|---|
| | | | | | mRNA |
| | | | | | expression |
| Cell activity | Expressed | mRNA expression level (% of GAPDH) | | | level (FR^{High} |
| (mMES) | genes | Input cells | FR^{High} cells | FR^{Low} cells | to FR^{Low}) |
| Pluripotency | Oct4 | 24.16 | 23.81 | 19.51 | 1.22:1 |
| | Sox2 | 4.80 | 3.68 | 3.74 | 0.98:1 |
| | Nanog | 4.54 | 4.15 | 3.04 | 1.36:1 |
| | cMyc | 1.36 | 0.94 | 1.43 | 0.65:1 |
| | Klf2 | 8.87 | 11.86 | 4.82 | 2.46:1 |
| | Klf4 | 0.586 | 0.716 | 0.354 | 2.02:1 |
| | Rex1 | 10.19 | 9.45 | 5.02 | 1.88:1 |
| | Esrrb | 1.41 | 2.72 | 0.77 | 2.92:1 |
| Promotion of | Neurog1 | 0.4188 | 0.5448 | 0.0720 | 7.56:1 |
| neuronal | Neurod1 | 0.2140 | 0.1922 | 0.0888 | 2.16:1 |
| differentiation | Nkx2.2 | 0.00058 | 0.00072 | 0.00066 | 1.09:1 |
| | Ascl2 | 0.14680 | 0.18840 | 0.064685 | 2.91:1 |
| | Gfap | 0.1334944 | 0.154434 | 0.0148198 | 10.42:1 |
| | S100b | 0.0158646 | 0.0144152 | 0.00097501 | 1.48:1 |
| | Olig2 | 0.040732 | 0.0286779 | 0.0105885 | 2.71:1 |

iii) FreSH-Tracer-Based Analysis in Embryoid Bodies (EBs)

Embryoid bodies (EBs) were sorted based on the FR of the FreSH-tracer, and the genetic difference between the FR^{high} and FR^{low} subpopulations was analyzed in the same manner as described. The relative expression levels of pluripotency-related genes in FR^{high} cells compared with those in FR^{low} cells were analyzed. As shown in FIGS. 16 and 17, the relative expression levels of Neurog2, Olig2, T, Nkx2.5, Oct4, Esrrbb, Klf2, Klf4, cTnT, a-Actin, Mlc2v and Runx1 genes in FR^{high} cells compared with those in FR^{low} cells were analyzed, and the results are shown in Table 4 below.

TABLE 4

| | | | Relative mRNA expression | | |
|---|---|---|---|---|---|
| | | | level (FR^{High} to | | |
| | | Expressed | FR^{Low}) after EB formation | | |
| Cell activity | Cell type | genes | 0 days | 5 days | 7 days |
| Pluripotent | mESCs | Neurog2 | 2.06:1 | 3.59:1 | 2.62:1 |
| differentiation | | Olig2 | 1.42:1 | 0.79:1 | 2.74:1 |
| ability | | T | 0.31:1 | 2.09:1 | 0.07:1 |
| | | Nkx2.5 | 0.33:1 | 0.28:1 | 1.85:1 |
| | | Oct4 | 1.14:1 | 1.02:1 | 0.27:1 |
| | | Esrrbb | 3.35:1 | 0.03:1 | 0.04:1 |
| | | Klf2 | 2.78:1 | 0.06:1 | 0.13:1 |
| | | Klf4 | 2.29:1 | 0.48:1 | 0.42:1 |
| | | cTnT | 0.07:1 | 0.05:1 | 1.36:1 |
| | | a-Actin | 0.47:1 | 0.36:1 | 1.02:1 |
| | | Mlc2v | 1.86:1 | 0.05:1 | 0.83:1 |
| | | Runx1 | 0.18:1 | 0.38:1 | 0.78:1 |

TABLE 5

| | | | mRNA expression level (% of GAPDH) after EB formation | | | | | |
|---|---|---|---|---|---|---|---|---|
| | | | 0 days | | 5 days | | 7 days | |
| | Cell | Expressed | FR^{High} | FR^{Low} | FR^{High} | FR^{Low} | FR^{High} | FR^{Low} |
| Cell activity | type | genes | cells | cells | cells | cells | cells | cells |
| Pluripotent | mESCs | Neurog2 | 9.08E−05 | 4.41E−05 | 0.00038 | 0.00011 | 0.00087 | 0.00033 |
| differentiation | | Olig2 | 0.00532 | 0.00374 | 0.00065 | 0.00083 | 0.00762 | 0.00278 |
| ability | | T | 0.0403 | 0.1289 | 2.8934 | 1.3835 | 0.0577 | 0.8492 |
| | | Nkx2.5 | 0.0048 | 0.0145 | 0.0079 | 0.0279 | 0.0966 | 0.0523 |
| | | Oct4 | 19.823 | 17.382 | 1.4381 | 1.4091 | 0.0552 | 2.0402 |
| | | Esrrb | 3.0539 | 0.9127 | 0.0096 | 0.2757 | 0.0137 | 0.3606 |
| | | Klf2 | 11.329 | 4.0683 | 0.1809 | 3.0576 | 0.4648 | 3.503 |
| | | Klf4 | 0.66 | 0.2882 | 0.0748 | 0.1566 | 0.0795 | 0.1901 |
| | | cTnT | 0.001 | 0.0154 | 0.0224 | 0.4379 | 2.3416 | 1.7168 |
| | | a-Actin | 0.4085 | 0.8717 | 0.1752 | 0.4921 | 4.2195 | 4.1246 |
| | | Mlc2v | 0.0563 | 0.0302 | 0.1202 | 2.3777 | 11.91 | 14.301 |
| | | Runx1 | 0.0182 | 0.1017 | 0.0991 | 0.2626 | 0.1859 | 0.2385 |

All the primers used in RQ-PCR analysis for the above-described genes were designed using Primer Express 3.0 (Applied Biosystems), and the sequences thereof are shown in Table 6 below.

TABLE 6

| Primer names | Primer sequences(5′ → 3′) |
|---|---|
| hRunx2_qRT_F1 | TCTTAGAACAAATTCTCCCCTTT |
| hRunx2_qRT_R1 | TGCTTTGGTCTTGAAATCACA |
| hOCN_qRT_F1 | AGCAAAGGTGCAGCCTTTGT |
| hOCN_qRT_R1 | GCGCCTGGGTTCTCACT |
| hALP_qRT_FT | GACCTCCTCGGAAGAC1CTC |
| hALP_qRT_R1 | TGAAGGGCCTTGTCTGTG |
| hMSX2_qRT_F1 | CCCTCGAGCGCAAGTTCCGT |
| hMSX2_qRT_R1 | GGCGGGATGGGAAGCACAGG |
| hOct4_qRT_F3 | GAGCCCTGCACCGTCACC |
| hOct4_qRT_R2 | TTGATGTCCTGGGACTCCTCC |
| hS0x2_qRT_F3 | TACAGCATGTCCTACTCGCAGC |
| hSox2_qRT_R3 | GAGGAAGAGGCAACCACAGGGG |
| hStella_qRT_F3 | TTCGTACGCATGAAAGAAGACC |
| hStella_qRT_R2 | TCCCATCCATTAGACACGCAGA |
| hSDF1_qRT_Fl | TGCGTCCACGAGCTGTTTAC |
| hSDF1_qRT_Rl | CCCAAGGGAGTGTCAGGTAGAG |
| hCXCR4_qRT_F3 | ACTACACCGAGGAAATGGGCT |
| hCXCR4_qRT_R3 | CCCAGATGCCAGTTAAGAAGA |
| hHGF_qRT_F1 | CTCACACCCGCTGGGAGTAC |
| hHGF_qRT_R1 | TCCTTGACCTTGGATCCATTC |
| hcMET_qRT_Fl | AGCGTCAACAGAGGGACCT |
| hcMET_qRT_R1 | GCAGTGAACCTCCGACTGTATG |
| hMMP2_qRT_F2 | CCCACTGCGGTTTTCTCGAAT |
| hMMP2_qRT_R2 | CAAAGGGGTATCCATCGCCAT |
| hMMP9_qRT_F2 | GGGACGCAGACATCGTCATC |
| hMMP9_qRT_R2 | TCGTCATCGTCGAAATGGGC |
| hMMP12_qRT_F1 | GATCCAAAGGCCGTAATGTTCC |
| hMMP12_qRT_R1 | TGAATGCCACGTATGTCATCAG |
| hNFkB_qRT_Fl | AACAGAGAGGATTTCGYFICCG |
| hNFkB_ART_R1 | TTTGACCTGAGGTGACTTCT |
| hPDGFA_SCL_F1 | CCGCAGTGCACACCTAGAATG |
| hPDGFA_SCL_R1 | GCACACCAACAACACAGACAGA |
| hPDGFB_SCL_Fl | AGGCAACACTGCTGTCCACAT |
| hPDGFB_SCL_R1 | GTCCCACACCCACCTGGAA |
| hPDGFC_SCL_Fl | TCCAGCCATTACTAACCTATTCCTTT |
| hPDGFC_SCL_R1 | TATCAGGAAGCTGCCAAGTCTTTTT |
| hPDGFD_SCL_Fl | GCTGCAATAACCAGCAAGGTT |
| hPDGFD_SCL_R1 | AATATGCCTGCTTACATTTCAGCTAA |
| hPDGFRA_qRT_Fl | GAAGGCAGGCACATTTACA |
| hPDGFRA_qRT_Rl | GCGACAAGGTATAATGGCAGAAT |
| hPDGFRA_qRT_Fl | TGATGCCGACTAVATCT |
| hPDGFRA_qRT_Rl | TTTTCTTCTCGTGCAGTGTCAC |
| hTEK_qRT_Fl | GGAGACGGACCCAGCATTT |
| hTEK_qRT_R1 | CGGCAGCGAAGTGAAGGA |
| hTIE1_qRT_Fl | CCTGTGCCGAGCTCTATGAA_ |
| hTIFl_qRT_R1 | GCTCGTACACATCGTCACAGT |
| hVEGFA_SCL_F1 | CTTCTCTCTCCCTTCTGACA |
| hVEGFA_SCL_R1 | GGATGGCAGAGCTGAGTGTTAG |
| hVFEFB_SCL F1 | TCAGGGATAGCCCAGTCAATACA |
| hVEGFB_SCL_R1 | GCCACAGAAGGCTGTCTCCTT |
| hVEGFC SCL_F1 | AGTTCCACCACCAAACATGCA |
| hVECFC_SCL_R1 | CACTATATGAAAATCCTGGCTCACA |
| hVEGFRl_SCL_Fl | CTCTCTCCCTGATCGGTGACA |
| hVEGFR1_SCL_R1 | GGAGGGCAGAGCTGAGTGTTAG |
| hVEGFR2_SCL_F1 | GGTTGCATTACTGTACCCATCATTT |
| hVEGFR2_SCL_R1 | TGAGATGGAATCTGACCATGTTG |
| hANGPT1 gRT F1 | TGCTCACGTGGCTCGACTA |
| hANGPTl_qRT_R1 | AGCACAGCAACCTCAGCAGTTT |
| hANGPTl_qRT_F1 | GGTTTGATGCATGTGGTCCTT |
| hANGPT2_qRT_R1 | AATGCCGTTGAACTTATTGTGTTC |
| hTNFA_qR1_Fl | GCCAGGCAGGTTCTCTTCCT |
| hTNFA_qR1_Rl | TCAGTGCTCATGGTGTCCTTTC |
| hIFNG_qRT_Fl | CCAACGCAAAGCAATACATGA |
| hIFNG_qRT_Rl | TCCTTTTTCGCTTCCCTGTTTT |
| hCSF1_qRT_Fl | TGCTGGAGAAGGTCAAGAATGTC |
| hCSF1_qRT_Rl | GTTGTTGCAGTTCTTGCTGAAAA |
| hCSF2_qRT_F1 | AGCCCTGGGAGCATGTGA |
| hCSF2_qRT_R1 | ATTCATCTCAGCAGCAGTGTCTCT |
| hSTC1_qRT_Fl | CATGAGGCGCAGCAGAATGA |
| hSTC1_qRT_Rl | CAACGAACCACTTCAGCTGAGTT |
| hLIF_qRT_F1 | GAAAGCTTTGGTAGGTTCTTCGTT |
| hLIF_qRT_R1 | TGCAGGTCCAGCCATCAGA |
| hIDO1_qRT_F1 | TCCGTGAGTTGTCCTTTCAAA |
| hIDO1_qRT_R1 | CAGGCAGACCAGAGCTTTCACA |
| hIDO2_qRT_F1 | GATTGATGCTCACCAGCTTCAAG |
| hIDO2_qRT_R1 | GCTCCCGGTGACCCTTCAG |
| hMCP1_qRT_Fl | CAGCCAGATGCAATCAATGCC |
| hMCP1_qRT_Rl | TGGATCCTGAACCCACTTCT |
| hCXCL10_qRT_Fl | GTGGCATTCAAGGAGTACCTC |
| hCXCL10_qRT_Rl | TGATGGCCTTCGUCTGTGGATT |
| hiL1B_SCL F1 | GACAGAAACCACGGCCACAT |
| hiL1B_SCL R1 | TAAAGCGGTTGCTCATC |
| hiL1B_SCL F1 | GGGAGCCCCTTTGATGATTAAT |
| hiL10_SCL_R1 | GCCACAGCTTTCAAGAATGAAGT |
| hIL12A_SCL_Fl | TTCAGAATTCGGGCGTGACT |
| hIL12A_SCL_Rl | CCCCCTCCCTAGTTCTTAATCC |
| hIL12B_SCL_Fl | GCTATGGTGAGCCTTGATTGT |
| hIL12B_SCL_Rl | GCCATGGAAGCTGAA |
| hIL16_qRT_F1 | GCTGGTTAACTTGTTTGGCCTATT |
| hIL16_qRT_R1 | GGTGCCTCCAAGTTCTTGTCTAATT |
| hiL18_SCL_F1 | GCACTCCGGAGGTAGAGGTTGT |
| hiL18_SCL_R1 | TTTGAGATGGAGTTTTGCTGTTG |
| hATF2_qRT_F1 | AAGGTCATGGTAGCGGATTGG |
| hATF2_qRT_R1 | AGTGGATGTGGCTGGCTGTT |
| hHEY1_qRT_F1 | TACGGCAGGAGGGAAAGGTT |
| hHEY1_qRT_R1 | CCAGGCATTCCCGAAATCC |
| hFOSL_qRT_Fl | CCTGTACCTTGTATCTCCCTTTCC |
| hFOSL_qRT_Rl | AGTTAGGGAGGTGTGGTCATG |
| hFOSL2_qRT_F2 | CCTCGAACCTCGTCTTCCCTA |
| hFOSL2_qRT_R2 | TGAGCCTTGGAGGAGGATTC |
| hFSHB2_qRT_F2 | AGCTGTGAGCTGACCAACAT |
| hFSHB2_qRT_R2 | GTGTAGCAGTAGCCAGCACA |
| hJUN_qRT_F2 | GGATCAAGGCGGAGAGGAA |
| hJUN_qRT_R2 | GGGCGTTCTCTCCAGCTT |
| hJUNB_qRT_Fl | ACTCATACACAGCTACGGGATACG |
| hJUNB_qRT_Rl | CAGGCTCGGTTTCAGGAGTTT |
| hJUND_qRT_F1 | ACTTTTCTGGTCAGGGCTCG |
| hJUND_qRT_R1 | CGTTGCTGTTGCGGACAATC |
| hGRB_qRT_F1 | GCCATCGCCAAATATGACTTC |
| hGRB_qRT_R1 | TCGTTCAAAACCTTGAGGATGTC |
| hKITLG_qRT_F1 | TGAGAAAGGGAAGGCCAAAA |
| hKITLG_qRT_R1 | AGAGAAAACAATGCTGGCAATG |
| hMITF_qRT_Fl | GCCTCCAAGCCTCCGATAAG |
| hMITF_qRT_Rl | TGCATCTGCTCACGGATGAG |
| hOLR_qRT_Fl | CCTTTGATGCCCCACTTATTTAGA |
| hOLR_qRT_R1 | AACACCTCCTCGTGTATATATGCA |

Demonstration of Therapeutic Effects of FR^{High} Stem Cells in Asthma Mouse Model

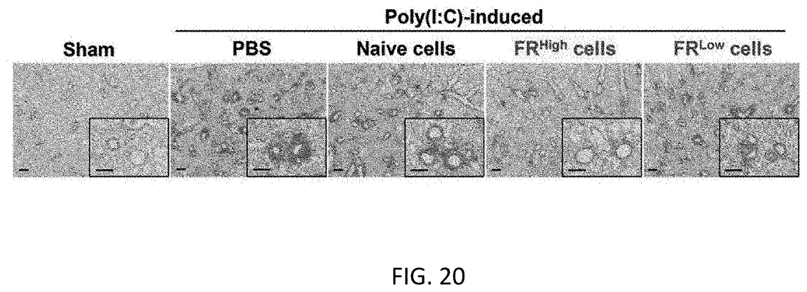

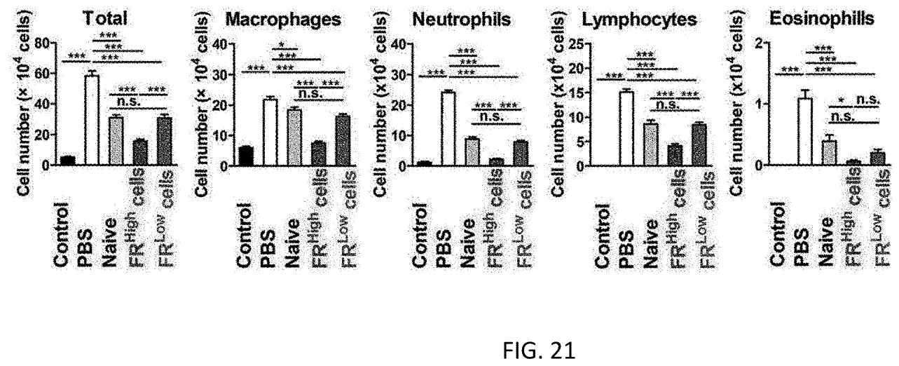

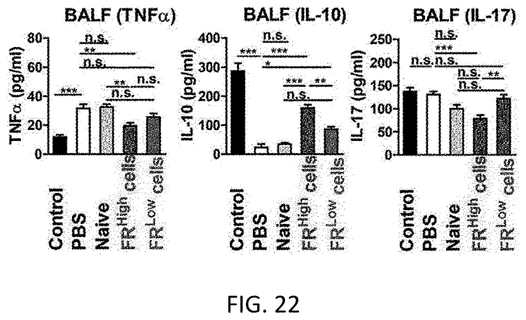

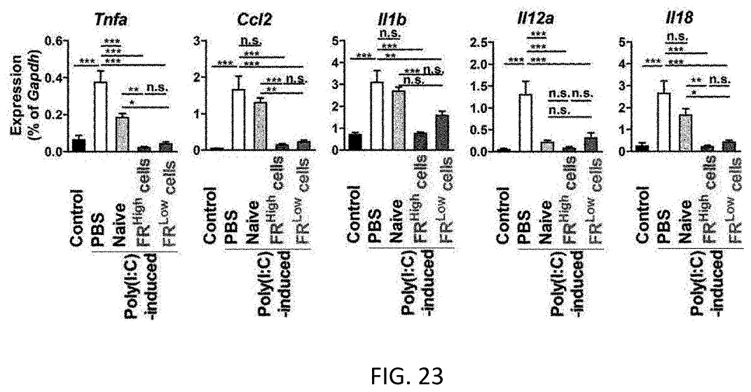

To confirm these results in vivo, the present inventors compared the therapeutic effects of FR^{High} and FR^{Low} hES-MSCs in a mouse model of virus-associated asthma. The mice were sensitized and challenged with ovalbumin and poly(I:C) and then injected with naïve hES-MSCs or the sorted or naive hES-MSCs via the tail vein (FIG. 19). Histological examination showed that inflammatory responses were markedly attenuated around the bronchial and perivascular areas in the lungs of the FR^{High} cell-injected mice compared with those of the FR^{Low} or naive cell-injected mice (FIG. 20). The number of inflammatory cells in the bronchoalveolar lavage fluid from FR^{High} cell-injected mice was smaller than that from FRFR^{Low} or naïve cell-injected mice (FIG. 21). Similarly, tumor necrosis factor-α and interleukin-17 (IL-17) levels were lower, whereas IL-10 levels were higher in the bronchoalveolar lavage fluid from FR^{High} cell-injected mice (FIG. 22). Quantitative reverse transcription-polymerase chain reaction (qRT-PCR) confirmed the significantly decreased mRNA levels of inflammatory cytokines in the lung tissues of FR_{High} cell-injected mice (FIG. 23). Moreover, immunohistochemical staining with human β2-microglobulin antibody showed a marked increase of engrafted cells in the lungs of FR^{High} hES-MSC-injected mice, which were identified as type-2 alveolar cells by staining with anti-prosurfactant protein C (SFTPC) antibody. These results indicated that the injected Hes-MSCs differentiated into the alveolar epithelium, contributing to tissue regeneration

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereof.

### INDUSTRIAL APPLICABILITY

The method for managing the quality of therapeutic stem cells which are provided according to the present invention can manage the quality of stem cells according to their characteristics based on an identified gene expression ratio and can selectively manage the necessary characteristics of the stem cells.

1-90. (canceled)

91. A therapeutic stem cell in which the expression level of any one or more selected from the group consisting of OCT4, SOX2, cMET, PDGFRA, PDGFRB, VEGF-R1 and VEGF-R2 is higher or lower than the expression level of GAPDH (glyceraldehyde 3-phosphate dehydrogenase) gene.

92. The therapeutic stem cell of claim 91, wherein the stem cell is any one type selected from the group consisting of mesenchymal stem cell (MSC), embryonic stem cell (ESC), or embryoid body (EB).

93. The therapeutic stem cell of claim 92, wherein the stem cell is a mesenchymal stem cell (MSC) in which the expression level of any one or more genes selected from the group consisting of OCT4, SOX2, cMET, PDGFRA, PDGFRB, VEGF-R1 and VEGF-R2 is lower than the expression level of the GAPDH (glyceraldehyde 3-phosphate dehydrogenase) gene.

94. The therapeutic stem cell of claim 92, wherein the stem cell is a mesenchymal stem cell (MSC) whose growth and proliferation are promoted when the expression level of cMET is 0.063- to 0.078-fold of that of GAPDH.

95. The therapeutic stem cell of claim 92, wherein the stem cell is a mesenchymal stem cell (MSC) whose engraftment rate, viability and vascular regeneration ability increase when the expression level of PDGFRA is 0.31- to 0.39-fold of that of GAPDH.

96. The therapeutic stem cell of claim 92, wherein the stem cell is a mesenchymal stem cell (MSC) whose engraftment rate, viability and vascular regeneration ability increase when the expression level of PDGFRB is 0.45- to 0.56-fold of that of GAPDH.

97. The therapeutic stem cell of claim 92, wherein the stem cell is a mesenchymal stem cell (MSC) whose engraftment rate, viability and vascular regeneration ability increase when the expression level of VEGF-R1 is 0.44- to 0.55-fold of that of GAPDH.

98. The therapeutic stem cell of claim 92, wherein the stem cell is a mesenchymal stem cell (MSC) whose engraftment rate, viability and vascular regeneration ability increase when the expression level of VEGF-R2 is 0.62- to 0.77-fold of that of GAPDH.

99. A method for treating asthma, the method comprising administering an effective amount of a pharmaceutical composition comprising the therapeutic stem cell according to claim 91 to a patient in need thereof.

100. A method for treating allergic asthma, the method comprising administering an effective amount of a pharmaceutical composition comprising the therapeutic stem cell according to claim 91 to a patient in need thereof.
